(19) United States
(12) Patent Application Publication
(10) Pub. No.: US 2022/0175007 A1
(43) Pub. Date: Jun. 9, 2022

(54) FASTING MIMICKING KETOGENIC DIET TO IMPROVE IMMUNE FUNCTION AND VACCINE RESPONSE AND MINIMIZE RISK IN ADULTS AND ELDERLY

(71) Applicant: UNIVERSITY OF SOUTHERN CALIFORNIA, Los Angeles, CA (US)

(72) Inventors: Valter D. LONGO, Playa del Rey, CA (US); Roberta BUONO, Newport Beach, CA (US)

(21) Appl. No.: 17/545,490

(22) Filed: Dec. 8, 2021

Related U.S. Application Data
(60) Provisional application No. 63/123,053, filed on Dec. 9, 2020.

(51) Int. Cl. A23L 33/00; A23L 23/00; A23L 25/00; A23L 7/126; A23L 2/60; A23L 19/00; A23F 3/16; A23L 33/155; A23L 33/16

(52) U.S. Cl. CPC A23L 33/30; A23L 23/00; A23L 25/00; A23L 7/126; A23V 2002/00; A23L 19/03; A23F 3/163; A23L 33/155; A23L 33/16; A23L 2/60

(57) ABSTRACT

A method for improving immune profile and function in adults and elderly is provided. The method includes a step of providing or administering a ketogenic fasting mimicking diet to a normal subject or a subject in need of immune profile and function improvement.

### CROSS-REFERENCE TO RELATED APPLICATIONS

This application claims the benefit of U.S. provisional application Ser. No. 63/123,053 filed Dec. 9, 2020, the disclosure of which is hereby incorporated in its entirety by reference herein.

### STATEMENT REGARDING FEDERALLY SPONSORED RESEARCH OR DEVELOPMENT

The invention was made with Government support under Contract No. HHSN271201400030C awarded by the National Institutes of Health (NIH). The Government has certain rights to the invention.

### TECHNICAL FIELD

The present invention, in general, relates to compositions and methods for a new fasting-mimicking ketogenic diet for improving immune profile and function. In particular, the present invention induces an increase/activation of white blood cells, lymphocytes, and hematopoietic stem cells. It has a higher regeneration capacity leading to efficient blood reconstitution and improved engraftment in the blood and bone marrow after transplantation. These results strongly support a role for FMD diets in stimulation of HSC self-renewal and improvement in both short- and long-term hematopoietic repopulation capacities; furthermore, it showed significant increases in antibody response against influenza virus.

### BACKGROUND

Intermittent or Periodic Fasting has been reported to promote lifespan extension and provide a range of protective effects, including neural stem cell generation in the nervous system and protection against chemotherapy side effects or cancer progression¹⁻⁵. Periodic Fasting Mimicking Diets (FMD) that allow individuals to maintain many of their normal dietary habits and do not cause excessive weight loss are becoming promising interventions to promote disease prevention and healthy aging.

Immunosenescence mainly involves a loss of adaptive immunity with relative preservation of innate immunity⁶, with a decreased function B cells and T cells. Lymphopoiesis declines with age and is associated with the transcriptional upregulation of genes that specify myeloid-lineage differentiation in hematopoietic stem cells, leading to altered production of immune cells and manifested in part as a shift in the lymphoid-to-myeloid ratio. Immunosenescence is a major factor responsible to increased severity of infections, poor response to vaccines, and higher cancer incidence seen in the elderly⁷. Old age is also one of the biggest risk factors for cancer, and immunosenescence plays a key role in both cancer development and the poor response to chemotherapy treatment^{8,9}. The decline in immune function is also responsible for the poor response to vaccination in the elderly with age and be predictor of the influenza vaccine response in humans as consistent with mouse data^{10,11}.

Accordingly, there is a need for an improved method for treating patients in need of improved immune function.

### SUMMARY

In at least one aspect, a highly nutritive 5 days ketogenic FMD regime to be consumed once a month for 2 months that will mimic the effects of fasting on age-related immunosenescence and enhance the effects of fasting by preventing or even reducing malnourishment and frailty is provided. This new formulation increases hematopoietic stem cell regeneration in the elderly and promotes stem cell self-renewal and improved immune profile and function.

In at least one aspect, a method for improving a subject's immune profile and/or function is provided. The method includes a step of administering an immune-enhancing ketogenic fasting-mimicking diet to a subject identified as needing or desiring improved immune profile and function.

In another aspect, an immune-enhancing ketogenic fasting-mimicking diet package for improving a subject's immune profile and/or function is provided. The immune-enhancing ketogenic fasting-mimicking diet package includes meal portions that include breakfast, lunch, and dinner portions for administering an immune-enhancing ketogenic fasting-mimicking diet for each day of a first time period. Characteristically, the meal portions provide the subject with at most 80 percent of a subject's normal calorie consumption per day with protein and sugar rejection.

In another aspect, the use of the immune-enhancing ketogenic fasting-mimicking diet package described herein for forming a medicament for improving immune profile and/or function is provided.

In another aspect, the use of the immune-enhancing ketogenic fasting-mimicking diet package described herein for improving a subject's immune profile and/or function is provided.

In still another aspect, a method for improving a subject's immune profile and/or function is provided. The method includes a step of receiving subject information for a subject identified as needing or desiring improved immune profile and function that includes a subject's weight and optionally their age. Meal portions are formed for each day of a predetermined time period during which an immune-enhancing ketogenic fasting-mimicking diet is administered. The meal portions provide a subject with at most 80 percent of a subject's normal calorie consumption per day of the immune-enhancing ketogenic fasting-mimicking diet with protein and sugar rejection.

In still another aspect, the meal portions are are scaled by a factor in accordance to a subject's weight.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIGS. 1A and 1B. Dietary intervention's effect on the numbers of WBCs (A, C) and LYs (B, D) N=4/group *p<0.05.

FIGS. 2A, 2B, and 2C. Flow cytometry analyses of bone marrow to identify long-term (LT) and short-term (ST) hematopoietic stem cells (HSCs) as well as the multipotent progenitors (MPPs) after two FMD cycles in 18 months old mice.

FIG. 3. Relative contribution of engraftment from the different cell sources was assessed by flow cytometry of the PB with CD45.2 (B6) and CD45.1 (B6.SJL) antigens. Data are shown as Mean±SEM *p<0.05, **p<0.01, ***p<0.001 vs control.

FIGS. 4A, 4B, and 4C. Lymphoid population assessed by flow cytometry of the PB from RF1D transplants with B220+ and CD3+, myeloid population assessed by Mac1 and GR1 staining after 4, 8, 12 and 16 weeks.

FIGS. 5A, 5B, and 5C. Flow cytometry of bone marrow to identify CD45.2+BM cell, total LSK and long-term (LT), short-term (ST) HSCs & multipotent progenitors (MPPs) at end of transplant. N=6. Mean±SEM, *p<0.05, **p<0.01, ***p<0.001 vs control.

FIGS. 6A, 6B, 6C, and 6D. Relative contribution of engraftment from the different cell sources was assessed by flow cytometry of the PB with CD45.2 (B6) and CD45.1 (B6.SJL) antigens after the second transplant. Lymphoid population assessed by flow cytometry of the PB with B220+ and CD3+, myeloid population assessed by Mac1 and GR1 staining after 4, 8, 12 and 16 weeks. Data are shown as Mean±SEM (N=6/group) *p<0.05, **p<0.01, ***p<0.001 vs control.

FIGS. 7A, 7B, and 7C. Flow cytometry of bone marrow to identify CD45.2+BM cell, total LSK and long-term (LT), short-term (ST) HSCs & multipotent progenitors (MPPs) at end of transplant after 4 months.

FIGS. 8A, 8B, 8C, 8D, 8E, and 8F. Antibody titers in sera collected at 28 days after vaccination by hemagglutination inhibition assay (HAI). Antibody titer and response rate (titer ≥40) against Fluzone HD 2017-2018 season vaccine (A-B) FR-1565 A/Michigan/45/2015 X-275 (H1N1) C-D), FR-1566 A/Hong Kong/4801/2014 17 X-263-B (H3N2) E-F), FR-1567 B/Brisbane/60/2008 (B Victoria lineage) (International Reagent Resource, IRR), N=6. Mean of antibody titers are shown. *p<0.05, **p<0.01, ****p<0.0001.

FIGS. 9A, 9B, and 9C. The lymphoid to myeloid ratio in human subjects that maintained a normal diet and subjects following 3 monthly cycles of a ketogenic fasting-mimicking diet. The lymphoid to myeloid ratio was increased for all participants that completed the FMD. The lymphoid to myeloid ratio at baseline for study participants older than 40 years at baseline (BL>40), after completion of one FMD cycle and 3-5 days of resuming normal food intake after the 3rd FMD cycle.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

Throughout this application, where publications are referenced, the disclosures of these publications in their entireties are hereby incorporated by reference into this application to more fully describe the state of the art to which this invention pertains.

The term “subject” refers to a human or other animal, including birds and fish as well as all mammals such as primates (particularly higher primates), horses, birds, fish sheep, dogs, rodents, guinea pigs, pig, cat, rabbits, and cows.

### Abbreviations

“EoD” means end of the diet.
“FMD” is a ketogenic fasting-mimicking diet.
“HSC” means hematopoietic stem cell.
“kcal” means kilocalorie.
“K/uL” means thousands per cubic milliliter.
“LT” means long-term.
“LY” means lymphocytes.
“MMP” means multipotent progenitors.
“PB” means peripheral blood.
“RF1d” means refeeding for 1 day.
“RF3d” means refeeding for 3 days.
“ST” means short-term.
“WBC” means white blood cell.

The terms “kilocalorie” (kcal) and “Calorie” refer to the food calorie. The term “calorie” refers to the so-called small calorie.

The term “subject” refers to a human or animal, including all mammals such as primates (particularly higher primates), sheep, dog, rodents (e g., mouse or rat), guinea pig, goat, pig, cat, rabbit, and cow.

The term “normal caloric intake (i.e., consumption)” means the number of kcal that a subject consumes per day to maintain his/her weight. In a refinement, a subject's normal caloric intake can be determined by interrogation or from the subject's weight. In another refinement, a subject's normal caloric intake per day is estimated from the formula:

$$normal caloric intake = \frac{2000 kcal}{180 lb} \times ( {subject}^{′} s weight in pounds )$$

which is estimated at 2000 kcal for a 180 pound subject.

It should be appreciated that if the FMD is administered for less than the number of days indicated in a range, the FMD is still administered in the amounts of the named days. For example, when a range is indicated as for day 1 and days 2 to 5 and any additional days, if the FMD is only administered for 3 days, the indicated amounts are provided for days 1, 2, and 3.

The term “fasting-mimicking diet” (FMD) means a diet that mimics the effects of fasting typically by providing a subject with at most 50% of their normal caloric intake per day but with some nutritional component so that fasting is mimicked while a subject is not completely starved. Typically, this diet is ketogenic since it causes an increase in ketone bodies and stimulates ketone body-based metabolism so it can also be referred to as a ketogenic FMD. In particular, FMD affects IGF-1, AKT and TOR signaling by regulation of growth hormone signaling upstream of IGF-1. It also affects the levels of glucose, the release of insulin, and the levels of the hunger hormone leptin. Low levels of IGF-1, leptin, insulin, and glucose and higher levels of ketone bodies and IGFBP1 cooperate to promote the effects of the FMD. Examples of useful fasting-mimicking and enhancing diets and methods for monitoring the effects of these diets on markers such as IGF-1 and IGFBP1 in the context of the present invention are set forth in U.S. patent application Ser. No. 14/273,946 filed May 9, 2014; Ser. No. 14/497,752 filed Sep. 26, 2014; Ser. No. 12/910,508 filed Oct. 22, 2010; Ser. No. 13/643,673 filed Oct. 26, 2012; Ser. No. 13/982,307 filed Jul. 29, 2013; Ser. No. 14/060,494 filed Oct. 22, 2013; Ser. No. 14/178,953 filed Feb. 12, 2014; Ser. No. 14/320,996 filed Jul. 1, 2014; Ser. No. 14/671,622 filed Mar. 27, 2015; the entire disclosure of these patent applications is hereby incorporated by reference. The fasting mimicking diet set forth in U.S. patent application Ser. Nos. 14/060,494 and 14/178,953 are found to be particularly useful in the present invention. Additional examples of FMD diets are found in U.S. patent application Ser. No. 15/148,251 and WIPO Pub. No. WO2011/050302 and WIPO Pub. No. WO2011/050302; the entire disclosures of which are hereby incorporated by reference. In a refinement, the FMD can be a low sodium diet that provides less than 1000 mg of sodium chloride per day.

In an embodiment, a method for treating a subject in need of immune profile and function improvement. The method includes a step of providing or administering an immune-enhancing ketogenic fasting-mimicking diet to a subject in need of immune profile improvement and/or function improvement. A subject's immune status can be evaluated by a medical professional (e.g., a medical doctor) from a subject's medical history, blood tests, and other specialized tests. Medical history can include frequency of flu or other respiratory infections, smoking status, history of cancer, weight, age, and the like. Blood tests (e.g., CDC) can determine the levels of blood cells and immune system cells as well as levels of infection-fighting proteins. In severe cases, a bone marrow biopsy can be performed. Immune profiling typically involves the analysis of T- and B-cell receptors that allow cells to bind specific antigens, proliferate, and then rapidly mount a specific immune response. Immune profiling can also involve the evaluation of immune marker as set forth in the experiments described below. Poor immune status can also be indicated by frequent digestive problems, fatigue, delayed wound healing, and skin infections.

In a variation, the immune-enhancing ketogenic fasting-mimicking diet is formulated to provide at most 80% of their normal caloric intake per day but with a nutritional component so that the subject is not starved. In some refinements, the immune-enhancing ketogenic fasting-mimicking diet is formulated to provide at most, in increasing order of preference, 80%, 75%, 70%, 65%, 60%, 55% or 50% of the subject's normal caloric intake per day. In a refinement, the FMD provides at most the following number of kcal:

$$maximum FMD caloric intake = \frac{2000 kcal}{180 lb} \times ( {subject}^{′} s weight in pounds ) \times 0.5 .$$

In a useful variation, the subject consumes (i.e., is provided with) less calories on day 1 than on days 2-5 or any additional days. In a variation, the fasting mimicking diet is an immune-enhancing fasting mimicking diet. The FMD can be administered for a first time period of 1, 2, 3, 4, 5, 6, 7, 8, 9, or 10 days.

In some variations, the subject is greater than or equal to 50 years old. In a refinement, the subject is greater than or equal to 60 years old. In another refinement, the subject is greater than or equal to 70 years old. In still another refinement, the subject is greater than or equal to 75 years old.

In a variation, the immune-enhancing fasting mimicking diet can be used to enhance response to flu vaccination in a 50-85 year old subject by administering the immune-enhancing fasting mimicking diet 3 to 6 months before the flu season with the exclusion of subjects that they are frail and underweight.

In general, the immune-enhancing ketogenic FMD is formulated to provide 500 to 1600 kcal on the days that the FMD is administered. In a variation, the immune-enhancing fasting mimicking diet provides 750 to 1250 kcal for day 1 and 1100 to 1500 kcal for days 2-5 and any additional days. In a refinement, the immune-enhancing fasting mimicking diet provides 850 to 1150 kcal for day 1 and 1200 to 1400 kcal for days 2-5 and any additional days. In some refinements, the FMD is formulated to provide at most 1800 kcal, 1500 kcal, 1400 kcal, 1300 kcal, or 1250 kcal on day 1. In a further refinement, the FMD is formulated to provide at least 300 kcal, 400 kcal, 500 kcal, 600 kcal, or 800 kcal on day 1. Similarly, for days 2, 3, 4, 5, or any additional days, the FMD is formulated to provide at most 1700, 1600, 1500 kcal, 1400 kcal, 1300 kcal, 1250 kcal, 1000 kcal, or 950 kcal for each of days 2, 3, 4, 5, or any additional days. In another refinement, the FMD is formulated to provide at least 200 kcal, 300 kcal, 400 kcal, 500 kcal, and 600 kcal or each of days 2, 3, 4, 5, or any additional days. In a refinement, the FMD is formulated to provide 2.0 to 12 kcal per day for each day of the FMD. In another refinement, the FMD is formulated to provide 2.4 to 7 kcal per day for each day of the FMD. In a further refinement, the FMD is formulate to provide less than or equal to 30 g of sugar (e.g., 1 to 30 g) for each day of the FMD, less than 30 g of proteins (e.g., 1 to 30 g) for each day of the FMD, 15-30 grams of monounsaturated fats for each day of the FMD, 2-10 g of polyunsaturated fats for each day of the FMD, and less than 12 g of saturated fats (e.g., 1 to 12) for each day of the FMD.

The immune-enhancing FMD is based on human foods that allow nourishment during periods of low-calorie consumption and high fat. In a variation, immune-enhancing FMD is formulated to provide 20 to 55% calories from carbohydrates, 2 to 12% calories from protein and 30 to 75% calories from fat. In a refinement, the immune-enhancing FMD is formulated to provide 20 to 40% calories from carbohydrates, 2 to 12% calories from protein and 50 to 75% calories from fat. In a further refinement, the source of calories is 32% calories from carbohydrates, 6% calories from protein and 62% calories from fat. The diet is mainly composed by nut bars, olives, kale crackers, vegetable soup, pumpkin soup, mushroom soup, tomato soup.

In a refinement, the breakdown of fat sources in the immune-enhancing FMD is approximately 35 to 50 weight % from coconut oil; 25 to 45 weight derived from nuts that are part of the energy bars (almond meal, macadamia nut butter, Pecan, Coconut, flaxseed meal and coconut oil); 5 to 25 weight % from flax seeds, sunflower seeds, cashews, sesame seeds that are ingredients of the crackers; about 2 10 weight % from vegetables (e.g., soups); and about 1 to 6 weight percent from olives. In a refinement, from 45 to 60 weight % of the fat is saturated fat (primarily from coconut oil which is rich in medium chain fatty acids).

Table 1 provides average values for calories, fast, total carbohydrates, sugar and protein for an example of the FMD. In a variation, the FMD provides for day 1 fat calories in an amount from about 40 to 80% of the total calories provided for day 1. For days 2, 3, 4, 5, and any additional day, the FMD provides fat calories in an amount from about 30 to 60% of the total calories provided for these days. In a refinement, the FMD provides for day 1 fat calories in an amount from about 50 to 70% of the total calories provided for day 1. For days 2, 3, 4, 5, and any additional day, the FMD provides fat calories in an amount from about 35 to 55% of the total calories provided for these days. In another refinement, the FMD provides for day 1 fat calories in an amount from about 55 to 65% of the total calories provided for day 1. For days 2, 3, 4, 5, and any additional day, the FMD provides fat calories in an amount from about 40 to 50% of the total calories provided for these days. Typically, the amount of carbohydrates on day 1 is greater than the amounts provided for days 2, 3, 4, 5, and any additional days. In a variation, the FMD provides for day 1, total carbohydrate calories in an amount from about 20 to 50% of the total calories provided for day 1. For days 2, 3, 4, 5, and any additional days, the FMD provides total carbohydrate calories in an amount from about 30 to 60% of the total calories provided for these days. In a refinement, the FMD provides for day 1, total carbohydrate calories in an amount from about 25 to 45% of the total calories provided for day 1. For days 2, 3, 4, 5, and any additional days, the FMD provides total carbohydrate calorie in an amount from about 35 to 50% of the total calories provided for these days. In a refinement, the FMD provides for day 1, total carbohydrate calories in an amount from about 30 to 40% of the total calories provided for day 1. For days 2, 3, 4, 5, and any additional days, the FMD provides total carbohydrate calorie in an amount from about 40 to 50% of the total calories provided for these days. Typically, the amount of carbohydrates on day 1 is less than the amounts provided for days 2, 3, 4, 5, and any additional days.

In a variation, the FMD provides for days 1, 2, 3, 4, 5, and any additional days, sugar calories in an amount from about 1 to 18% of the total calories provided for these days. In a refinement, the FMD provides for days 1, 2, 3, 4, 5, and any additional days, sugar calories in an amount from about 7 to 15% of the total calories provided for these days. In a refinement, the FMD provides for days 1, 2, 3, 4, 5, and any additional days, sugar calories in an amount from about 9 to 13% of the total calories provided for these days. In a variation, the FMD provides for days 1, 2, 3, 4, 5, and any additional days, protein calories in an amount from about 1 to 18% of the total calories provided for these day. In a refinement, the FMD provides for days 1, 2, 3, 4, 5, and any additional days, protein calories in an amount from about 7 to 15% of the total calories provided for these days. In a refinement, the FMD provides for days 1, 2, 3, 4, 5, and any additional days, protein calories in an amount from about 9 to 13% of the total calories provided for these days.

TABLE 1
The macronutrient content for each day of the 5 day FMD.

| | | Day 2, 3, 4, 5 |
|---|---|---|
| | | and any |
| | | additional |
| | Day 1 | days* |
| Total | 1.066 | ~746 |
| Calorie | | |
| Fat | 59% | ~45% |
| Carbohydrate | 33% | ~46% |
| Sugar | 11% | ~10% |
| Protein | 9% | ~9% |

*Average values

In one variation of the immune-enhancing ketogenic fasting-mimicking, for day 1, total calorie consumption is 3.5-5 kcal per pound (or 7.7-11 kcal per kilogram). The daily diet, to be fasting-mimicking and ketogenic, should contain less than or equal to 25 g of sugar (e.g., 1 to 25 g), less than 23 g of proteins (e.g., 1 to 23 g), 16-25 grams of monounsaturated fats, 4.8-8 g of polyunsaturated fats, and 1-10 g of saturated fats. For days 2, 3, 4, 5, or any additional days, total calorie consumption is 2.4-4 kcal per pound (or 5.3-8.8 kcal per kilogram). For days 2, 3, 4, 5, or any additional days, the diet should contain less than 16 g of sugars (e.g., 1 to 16 g of sugars), less than 15 g of protein (e.g., 1 to 15 g of protein), 8-12 g of monounsaturated fats, 2-4 g of polyunsaturated fats and 1-6 grams of saturated fats. Diet should also be high nourishment containing 100% of the RDA for vitamins, minerals, and essential fatty acids.

In another variation, the immune-enhancing ketogenic fasting-mimicking diet provides the subject with 4.5 to 7 kcal/pound of body weight/day on day 1 followed by 3 to 5 kcal/pound of body weight/day for days 2, 3, 4, 5, or any additional days. In a refinement, the immune-enhancing ketogenic fasting-mimicking diet provides the subject with 7 kcal/pound of body weight/day on day 1 followed by 4 kcal/pound of body weight/day for days 2, 3, 4, 5, or any additional days. In another variation, the immune-enhancing ketogenic fasting-mimicking diet provides the subject with 3-6 kcal/pound of body weight/day on day 1 followed by 2-4 kcal/pound of body weight/day for days 2-4.

In another variation, the FMD is formulated to provide less than 30 g of sugar on day 1 and less than 20 g of sugar on days 2, 3, 4, 5, or any additional days. The diet should contain less than 28 g of proteins on day 1 and less than 18 g of proteins on days 2, 3, 4, 5, or any additional days, mostly or completely from plant-based sources. The diet should contain between 20 and 30 grams of monounsaturated fats on day 1 and 10-15 grams of monounsaturated fats on days 2, 3, 4, 5, or any additional days. The diet should contain between 6 and 10 grams of polyunsaturated fats on day 1 and 3-5 grams of polyunsaturated fats on days 2, 3, 4, 5, or any additional days. The diet should contain less than 12 g of saturated fats on day 1 and less than 6 grams of saturated fats on days 2, 3, 4, 5, or any additional days. Typically, the fats on all days are derived from a combination of the following: almonds, macadamia nuts, pecans, coconut, coconut oil, olive oil, and flaxseed. In a refinement, the FMD diet includes over 50% of the recommended daily value of dietary fiber on all days. In a further refinement, the amount of dietary fiber is greater than 15 grams per day on all five days or any additional days. The diet should contain 12-25 grams of glycerol per day on days 2, 3, 4, 5, or any additional days. In a refinement, glycerol is provided at 0.1 grams per pound body weight/day.

In some variations, the FMDs set forth herein are formulated to provide at least 60% calories from fatty acids and/or 2-5% calories from glycerol and/or up-to 5% of calories from plant-based proteins, and/or a maximum of 60% of calories from carbohydrates (these amounts are with respect to the total calories provided by the FMD. In some refinements, the FMD is formulated to provide a maximum of, in increasing order of preference, 15%, 12%, 10%, 8%, 6%, or 5% calories from plant-based proteins. In some refinements, the FMD is formulated to provide a maximum, in increasing order of preference, 65%, 60%, 55%, 50%, 60%, 35% calories from carbohydrates of the total calories provided by the FMD. Advantageously, carbohydrates are complex carbohydrates from plant sources such as soy, rice, or other grains. It should be appreciated that typically the amount of plant-based proteins and carbohydrates are greater than 1% of the total calories provided by the FMD At least a portion of the fats on all days are derived from a combination of the following: almonds, macadamia nuts, pecans, coconut, coconut oil, olive oil and flaxseed. In a refinement, at least 50% of the calories from fatty acids are from coconut oil and tree nuts (e.g., macadamia nuts, walnuts, or almonds). Additional fat sources include vegetable oil such as soybean oil. In a further refinement, the low protein diet incudes fat sources such that at least 25 percent of calories from fat are short-chain fatty acids having from 2 to 7 carbon atoms and/or from medium-chain saturated fatty acids having from 8 to 12 carbon atoms. Specific examples of fatty acids include lauric and/or myristic acid, and fat sources include olive oil, kernel oil and/or coconut oil. In another refinement, the immune-enhancing ketogenic fasting-mimicking diet includes calories from fat in an amount from about 0 to 22 percent of the total calories contained in the diet.

Advantageously, the FMD diet can be formulated to provide over 50% of the recommended daily value of dietary fiber on all days. In a refinement, the amount of dietary fiber is greater than 15 grams per day on all five days or any additional days. In some variations, the immune-enhancing ketogenic fasting-mimicking diet includes 2 to 5% calories from glycerol. The FMD can be formulated to provide 12-25 grams of glycerol per day on days 2, 3, 4, 5, or any additional days. In a refinement, glycerol is provided at 0.1 grams per pound body weight (of the subject)/day.

In some variations, the FMDs set forth herein are formulated to provide the following micronutrients (at least 95% non-animal based): over 5,000 IU of vitamin A per day (days 1-5 or any additional days.); 60-240 mg of vitamin C per day (days 1-5 or any additional days); 400-800 mg of Calcium per day (days 1-5 or any additional days); 7.2-14.4 mg of Iron per day (days 1-5 or any additional days); 200-400 mg of Magnesium per day (days 1-5 or any additional days); 1-2 mg of copper per day (days 1-5 or any additional days); 1-2 mg of Manganese per day (days 1-5 or any additional days); 3.5-7 mcg of selenium per day (days 1-5 or any additional days); 2-4 mg of Vitamin B1 per day (days 1-5 or any additional days); 2-4 mg of Vitamin B2 per day (days 1-5 or any additional days); 20-30 mg of Vitamin B3 per day (days 1-5 or any additional days); 1-1.5 mg of Vitamin B5 per day (days 1-5 or any additional days); 2-4 mg of Vitamin B6 per day (days 1-5 or any additional days); 240-480 mcg of Vitamin B9 per day (days 1-5 or any additional days); 600-1000 IU of Vitamin D per day (days 1-5 or any additional days); 14-30 mg of Vitamin E per day (days 1-5 or any additional days); over 80 mcg of Vitamin K per day (days 1-5 or any additional days); 16-25 mcg Vitamin B12 are provided during the entire 5-day period or any additional days; 600 mg of Docosahexaenoic acid (DHA, algae-derived) are provided during the entire 5-day period or any additional days. The FMD diet provides high micronutrient content mostly (i.e., greater than 50 percent by weight) from natural sources including: Kale, Cashews, Yellow Bell Pepper, Onion, Lemon Juice, Yeast, Turmeric. Mushroom, Carrot, Olive Oil, Beet Juice, Spinach, Tomato, Collard, Nettle, Thyme, Salt, Pepper, Vitamin B12 (Cyanocobalamin), Beets, Butternut Squash, Oregano, Tomato Juice, Orange Juice, Celery, Romaine Lettuce, Cumin, Orange Rind, Citric Acid, Nutmeg, Cloves, and combinations thereof. These micronutrients can be provided in the FMD as a micronutrient supplement composition. The micronutrient supplement composition in these meal plans is a vegetable powder with vitamins and minerals supplements. Table 2 provides an example of additional micronutrient supplementation that can be provided in the FMD diet.

TABLE 2
Micronutrient Supplementation

| | | | | Amount | |
|---|---|---|---|---|---|
| | Supplement | Formula | Amount | Range | Unit |
| Vitamin A | | | 1250 IU | 900-1600 | IU |
| Vitamin C | Ascorbic Acid | C₆H₈O₆ | 15.0000 | 10-20 | mg |
| Ca | Calcium Carbonate | CaCO₃ | 80.0000 | 60-100 | mg |
| Fe | Ferrous Fumarate | C₄H₂FeO₄ | 4.5000 | 3-6 | mg |
| Vitamin D3 | Cholecalciferol | C₂₇H₄₄O | 0.0025 | 0.001-0.005 | mg |
| Vitamin E | dl-Alpha Tocopheryl Acetate | C₂₉H₅₀O₂ | 5.0000 | 3-7 | mg |
| Vitamin K | Phytonadione | | 0.0200 | 0.1-0.04 | mg |
| Vitamin B1 | Thiamine Mononitrate | C₁₂H₁₇N₅O₄S | 0.3750 | 0.15-0.5 | mg |
| Vitamin B2 | Riboflavin E101 | C₁₇H₂₀N₄O₆ | 0.4250 | 0.2-0.6 | mg |
| Vitamin B3 | Niacinamide | C₆H₆N₂O | 5.0000 | 3-7 | mg |
| Vitamin B5 | Calcium Pantothenate | C₁₈H₃₂CaN₂O₁₀ | 2.5000 | 1.5-4.0 | mg |
| Vitamin B6 | Pyridoxine Hydrochloride | C₈H₁₁NO₃•HCl | 0.5000 | 0.3-0.7 | mg |
| Vitamin B7 | Biotin | C₁₀H₁₆N₂O₃S | 0.0150 | 0.01-0.02 | mg |
| Vitamin B9 | Folic Acid | C₁₉H₁₉N₇O₆ | 0.1000 | 0.07-0.14 | mg |
| Vitamin B12 | Cyanocobalamin | C₆₃H₈₈CoN₁₄O₁₄P | 0.0015 | 0.001-0.002 | mg |
| Cr | Chromium Picolinate | Cr(C6H4NO2)3 | 0.0174 | 0.014-0.022 | mg |
| Cu | Cupric Sulfate | CuSO4 | 0.2500 | 0.18-0.32 | mg |
| I | Potassium Iodide | KI | 0.0375 | 0.03-0.045 | mg |
| Mg | Magnesium Oxide | MgO | 26.0000 | 20-32 | mg |
| Mn | Manganese Sulfate | MnSO₄ | 0.5000 | 0.3-0.7 | mg |
| Mo | Sodium Molybdate | Na₂MoO₄ | 0.0188 | 0.014-0.023 | mg |
| Se | Sodium Selenate | Na₂O₄Se | 0.0175 | 0.014-0.023 | mg |
| Zn | Zinc Oxide | ZnO | 3.7500 | 3-5 | mg |

It should be appreciated, that the FMD can be formulated to within plus/minus 20 percent of the values indicated in Table 2.

In some variations, the FMD includes caloric restriction with respect to proteins and sugars (e.g., glucose). In one refinement, the immune-enhancing ketogenic fasting-mimicking diet includes protein in an amount that is less than 15 percent of total calories provided by the immune-enhancing ketogenic fasting-mimicking diet. In a further refinement, the immune-enhancing ketogenic fasting-mimicking diet includes protein in an amount that is at most, in increasing order of preference, 15%, 12%, 10%, 8%, or 5% of total calories provided by the immune-enhancing ketogenic fasting-mimicking diet and in an amount that is at least 0%, 2%, 3%, 5%, or 6% of total calories provided by the immune-enhancing ketogenic fasting-mimicking diet. In another refinement, the immune-enhancing ketogenic fasting-mimicking diet includes sugars in an amount that is less than 15 percent of the total calories provided by the immune-enhancing ketogenic fasting-mimicking diet. In a further refinement, the immune-enhancing ketogenic fasting-mimicking diet includes sugars (e.g., glucose) in an amount that is at most, in increasing order of preference, 15%, 12%, 10%, 8%, or 5% of total calories provided by the immune-enhancing ketogenic fasting-mimicking diet and in an amount that is at least, in order of preference, 0%, 2%, 3%, 5%, or 6% of total calories provided by the immune-enhancing ketogenic fasting-mimicking diet.

An example of a useful FMD package is provided in US Pat Pub No. 20180228198; the entire disclosure of which is hereby incorporated by reference. Such FMDs can include soups, nutrition bars, crackers (e.g., kale crackers), an optional energy drink with a glucose substitute, olives, vitamin and mineral supplements (e.g., with a vegetable powder), algal oil, and optional teas. The FMD can include nutrition bars, and soups as meals to be consumed for breakfast, lunch, and dinner. Examples of soup compositions are disclosed in US Pat Pub No. 20180228198; the entire disclosure of which is hereby incorporated by reference. Examples of nutrition bars are disclosed in US Pat Pub No. 20180228198; U.S. patent application Ser. No. 16/680,911 filed Nov. 12, 2019; and U.S. Pat. No. 17,347,212, filed Jun. 14, 2021 the entire disclosures of which are hereby incorporated by reference. Examples of nutrition bars are disclosed in US Pat Pub No. 20180228198; and U.S. Pat. No. 17,347,212, filed Jun. 14, 2021 the entire disclosures of which are hereby incorporated by reference. It should be appreciated that each of the soup, broth, tea, and energy drink compositions set forth herein can be in powder or solid form and are designed to have added water when consumed.

Typically, the FMD provides one or more FMD soups on each day of the diet. Each FMD soup can be formulated to provide less than about 1 gram of saturated fat per serving, less than about 5 grams of trans fat per serving (optimally, less than 1 gram of trans fat), less than about 10 grams of protein per serving, less than about 40 grams of carbohydrates per serving.

In a variation, each FMD soup is formulated to provide from about 80 kcal to 150 kcal preserving. In the context of formulations, “provide” also means “includes.” In a refinement, each FMD soup is formulated to provide about 1 to 5 grams of total fat per serving. In a further refinement, each FMD soup is formulated to provide less than about 1 grams of saturated fat per serving and less than about 5 grams of trans fat (optimally, less than 1 gram of trans fat). In a refinement, each FMD soup is formulated to provide less than about 10 grams of protein per serving. In a further refinement, each FMD soup is formulated to provide from about 1 to 8 grams of protein per serving. In a further refinement, each FMD soup is formulated to provide from about 2 to 6 grams of protein per serving. In still another refinement, each FMD soup is formulated to provide less than about 40 grams of carbohydrates per serving. In a further refinement, each FMD soup is formulated to provide from about 10 to 35 grams of carbohydrates per serving. In still another refinement, each FMD soup is formulated to provide from about 20 to 30 grams of carbohydrates per serving.

The FMD soups can be formulated with in these limits from any combination of components selected from the group consisting of black beans, butternut squash, quinoa, tomatoes, mushrooms, white beans (e.g., great northern beans), brown beans, spinach, green tea extract, rice flour, onions, brown rice powder, carrots, inulin, leeks, olive oil, cabbage, potatoes, olives, peas, pumpkin, maltodextrin, and celery, chicory root fiber, sea salt, yeast, basil, parsley, garlic, rosemary extract, coriander, oregano, potato starch, potato flakes, zucchini squash, turmeric. In a refinement, the FMD soup can be formulated with in these limits from a first component selected from the group consisting of black beans, butternut squash, quinoa, tomatoes, mushrooms, white beans (e.g., great northern beans), brown beans, spinach, and combinations thereof and a second component selected from the group consisting of green tea extract, rice flour, onions, brown rice powder, carrots, inulin, leeks, olive oil, cabbage, potatoes, olives, peas, pumpkin, maltodextrin, and celery, chicory root fiber, sea salt, yeast, basil, parsley, garlic, rosemary extract, coriander, oregano, potato starch, potato flakes, zucchini squash, turmeric, and combinations thereof. Typically, the FMD soups are provided as a powder that can be combined with water to form the soup, typically, with heating.

Examples of soups includes vegetable soups, quinoa-containing minestrone soups, mushroom-containing soup, tomato-containing soup composition, bean-containing minestrone soup composition, pumpkin soup composition, white bean and spinach soup, back bean soup, butternut squash soup, butternut squash and quinoa soup, and a vegetable broth. A specific example of a vegetable soup composition includes onions, tomatoes, spinach, green tree extract, optionally rice flour, optionally brown rice powder, optionally carrots, leeks, and optionally inulin. A specific example of a quinoa-containing minestrone soup composition includes quinoa, green tea extract, optionally olive oil, optionally cabbage, optionally potatoes, optionally rice flour, and optionally tomatoes and optionally no turmeric. A specific example of a vegetable soup composition includes carrots, inulin, leeks, onions and rice flour. A specific example of a mushroom-containing soup composition includes mushrooms, green tea extract, optionally brown rice powder, optionally carrots, and optionally inulin. A specific example of a mushroom-containing soup composition includes carrots, inulin, mushrooms, onions, and rice flour. A specific example of a tomato-containing soup composition includes tomatoes, green tea extract, optionally inulin, and optionally onions. A specific example of a tomato-containing soup composition includes tomatoes, inulin, olives, onions, potatoes, and rice flour. A specific example of a quinoa-containing minestrone soup composition includes quinoa, green tea extract, optionally olive oil, optionally cabbage, optionally potatoes, optionally rice flour, and optionally tomatoes and optionally no turmeric. A specific example of a quinoa-containing minestrone soup composition includes quinoa, green tea extract, optionally olive oil, optionally cabbage, optionally potatoes, optionally rice flour, and optionally tomatoes and optionally no turmeric. A specific example of a bean-containing minestrone soup composition includes white beans (e.g., great northern beans), great tea extract, optionally cabbage, and optionally potatoes. In a refinement, the bean-containing minestrone soup composition includes brown beans, carrots, peas, potato, and rice flour. A specific example of a pumpkin soup composition includes pumpkin, green tree extract, optionally rice flour, optionally carrots, and optionally brown rice powder. A specific example of a first vegetable broth includes carrots, maltodextrin, celery, spinach, and tomatoes. A specific example of a vegetable broth includes carrots, maltodextrin, celery, spinach, and tomatoes. A specific example of a white bean and spinach soup composition includes white beans, whole grain brown rice, rice flour, chicory root fiber, olive oil, spinach leaves, sea salt, celery, onion, yeast extract, and garlic. A specific example of a black bean soup composition includes black beans, chicory root fiber, whole grain brown rice, olive oil, potato starch, rosemary extract, sea salt, onion, yeast extract, garlic, coriander, and oregano. A specific example of a butternut squash composition includes butternut squash, rice flour, whole grain brown rice, chicory root fiber, sea salt, carrots, olive oil, potato starch, rosemary extract, onion, and yeast extract. A specific example of a butternut squash and quinoa composition includes butternut squash, quinoa, whole grain brown rice, rice flour, chicory root fiber, olive oil, potato starch, rosemary extract, sea salt, onion, yeast extract, garlic, and chives.

In a variation, a first FMD nutrition bar is formulated to provide from about 150 kcal to 350 kcal per serving. In a refinement, the first FMD nutrition bar is formulated to provide about 10 to 35 grams of total fat per serving. In a further refinement, the first FMD nutrition bar is formulated to provide less than about 8 grams of saturated fat per serving and optionally, less than about 5 grams of trans fat (optimally, less than 1 gram of trans fat). In a refinement, each FMD nutrition bar is formulated to provide less than about 10 grams of protein per serving. In a further refinement, the first FMD nutrition bar is formulated to provide from about 1 to 8 grams of protein per serving. In a further refinement, the first FMD nutrition bar is formulated to provide from about 2 to 6 grams of protein per serving. In still another refinement, the first FMD nutrition bar is formulated to provide less than about 30 grams of carbohydrates per serving. In a further refinement, the first FMD nutrition bar is formulated to provide from about 8 to 25 grams of carbohydrates per serving. In still another refinement, the first FMD nutrition bar is formulated to provide from about 10 to 18 grams of carbohydrates per serving. The first FMD nutrition bar can be formulated within these limits with any combination of macadamia nuts, honey, pecans, almonds, almond butter, coconut flour, sea salt, mixed tocopherols (vitamin E), citric acid, and ascorbic acid.

In a variation, a second FMD nutrition bar is formulated to provide from about 70 kcal to 120 kcal per serving. In a refinement, the second FMD nutrition bar is formulated to provide about 3 to 10 grams of total fat per serving. In a further refinement, the second FMD nutrition bar is formulated to provide less than about 5 grams of saturated fat per serving and optionally, less than about 5 grams of trans fat (optimally, less than 1 gram of trans fat). In a refinement, each FMD nutrition bar is formulated to provide less than about 6 grams of protein per serving. In a further refinement, the second FMD nutrition bar is formulated to provide from about 1 to 6 grams of protein per serving. In a further refinement, the second FMD nutrition bar is formulated to provide from about 2 to 5 grams of protein per serving. In still another refinement, the second FMD nutrition bar is formulated to provide less than about 20 grams of carbohydrates per serving. In a further refinement, the second FMD nutrition bar is formulated to provide from about 5 to 18 grams of carbohydrates per serving. In still another refinement, the second FMD nutrition bar is formulated to provide from about 10 to 15 grams of carbohydrates per serving. The second FMD nutrition bar can be formulated within these limits with any combination of inulin, almond butter, brown rice crispy, cocoa powder, almonds, chocolate chips (cane sugar, unsweetened chocolate, cocoa butter), rolled oats, brown rice syrup, flaxseed oil, rice dextrin, grape juice, and salt.

In a refinement, the energy drink composition includes glycerin preferably in an amount of 20 to 60 weight %; water (purified) preferably in an amount of 40 to 80 weight %.

In another refinement, the drink powder composition includes total fat in an amount that provides from about 300 to about 700 kcal per 100 grams of the drink powder or ready to drink composition; carbohydrates in an amount that provides from about 50 to 150 kcal per 100 grams of the drink powder or ready to drink composition; and proteins in an amount that provides from about 30 to about 60 kcal per 100 grams of the drink powder composition or ready to drink composition.

In another embodiment, a fasting drink is provided. The fasting drink is typically formed from a fasting drink powder composition which is dissolved in water. In a refinement, the fasting drink and/or the fasting drink powder composition includes total fat in an amount that provides from about 200 to about 600 kcal per 100 grams of the fasting drink and/or the fasting drink powder composition; saturated fat in an amount from about 40 to 70 kcal per 100 grams of the fasting drink and/or the fasting drink powder composition; carbohydrates in an amount that provides from about 100 to 200 kcal per 100 grams of the fasting drink and/or the fasting drink powder composition; and proteins in an amount that provides from about 30 to about 60 kcal per 100 grams of the fasting drink and/or the fasting drink powder composition.

In another variation, the fasting drink and/or the fasting drink powder composition includes fat in an amount of at least, in increasing order of preference, 30 weight percent, 40 weight percent, 45 weight percent, or 50 weight percent and fat in an amount of at most, in increasing order of preference, 70 weight percent, 65 weight percent, 60 weight percent, or 55 weight percent. In a refinement, the fasting drink and/or the fasting drink powder composition includes saturated fat in an amount of at least, in increasing order of preference, 8 weight percent, 5 weight percent, 2 weight percent, or 1 weight percent and saturated fat in an amount of at most, in increasing order of preference, 20 weight percent, 15 weight percent, 10 weight percent, or 8 weight percent. The fasting drink and/or the fasting drink powder composition also includes carbohydrates in an amount of at least, in increasing order of preference, 1 weight percent, 3 weight percent, 10 weight percent, 15 weight percent, 20 weight percent, or 25 weight percent and carbohydrates in an amount of at most, in increasing order of preference, 35 weight percent, 30 weight percent, 40 weight percent, 38 weight percent, 35 weight percent, or 30 weight percent. The fasting drink and/or the fasting drink powder composition also includes proteins in an amount of at least, in increasing order of preference, 1 weight percent, 2 weight percent, 3 weight percent, 5 weight percent, or 8 weight percent and protein in an amount of at most, in increasing order of preference, 20 weight percent, 18 weight percent, 17 weight percent, 16 weight percent, or 15 weight percent. Unless stated to the contrary all percentages are weight percentages of the total weight of the fasting drink and/or the fasting drink powder composition. The fats used for the fasting drink and/or the fasting drink powder composition includes midchain triglycerides and/or monosaturated fat and/or and polyunsaturated fat. Suitable sources of monosaturated fat, but are not limited to, peanut butter, olives, nuts (e.g., almonds, pecans, pistachios, cashews), avocado, seeds (e.g., sesame), oils (e.g., olive, sesame, peanut, canola), etc. Suitable sources of polyunsaturated fat include, but are not limited to, walnuts, seeds (e.g., pumpkin, sunflower), flaxseed, and oils (e.g., safflower, soybean, corn).

In a refinement, the second vegetable broth (chicken flavoring) includes carrots, chicken flavoring, maltodextrin, celery, spinach, soy lecithin, and tomatoes. In a refinement, the second vegetable broth composition includes carrot (dehydrated, puffed, powder, pieces) preferably in an amount of 3 to 10 weight %; celery preferably in an amount of 3 to 12 weight %; garlic preferably in an amount of 3 to 9 weight %; maltodextrin preferably in an amount of 8 to 25 weight %; oil (canola) preferably in an amount of 0.5 to 2 weight %; onion preferably in an amount of powder, minced) preferably in an amount of 3 to 12 weight %; parsley preferably in an amount of 3 to 10 weight %; potato preferably in an amount of 1 to 6 weight %; salt preferably in an amount of 8 to 25 weight %; soy lecithin preferably in an amount of 0.5 to 3 weight %; spinach (leaf, powder) preferably in an amount of 3 to 12 weight %; tomatoes (fruit powder, sun dried, granules) preferably in an amount of 6 to 18 weight %; xanthan gum preferably in an amount of 0.5 to 4 weight %; and yeast extract preferably in an amount of 4 to 12 weight %.

In a refinement, the energy drink composition includes glycerin preferably in an amount of 20 to 60 weight %; water (purified) preferably in an amount of 40 to 80 weight %.

In a refinement, the tea composition that includes spearmint includes spearmint leaves organic preferably in an amount of 70 to 100 weight %.

In a refinement, the tea composition that includes lemon and spearmint includes lemon myrtle organic preferably in an amount of 3 to 12 weight %; lemon peel organic preferably in an amount of 10 to 25 weight %; spearmint leaves organic preferably in an amount of 50 to 95 weight %.

In a refinement, the tea composition that includes hibiscus includes hibiscus tea leaves organic preferably in an amount of 80 to 100 weight %.

In a refinement, the algal oil composition includes schizocatrium algae oil (DHA Omega-3) preferably in an amount of 80 to 100 weight %.

In a refinement, the nutrient replenishment composition (NR-1) includes beet root powder, calcium carbonate, carrots, collard leaf, kale leaf, and tomatoes. In a refinement, the nutrient replenishment composition (NR-1) includes ascorbic acid preferably in an amount of 1 to 3 weight %; beet root powder preferably in an amount of 6 to 20 weight %; beta carotene preferably in an amount of 0.05 to 0.15 weight %; calcium carbonate preferably in an amount of 6 to 20 weight %; carrot (dehydrated, puffed, powder, pieces) preferably in an amount of 6 to 20 weight %; cholecalciferol preferably in an amount of 0.00 weight %; chromium Picolinate preferably in an amount of 0.00 weight %; collard leaf powder preferably in an amount of 6 to 20 weight %; cupric sulfate preferably in an amount of 0.01 to 0.06 weight %; cyanocobalamin, 0.00; Dl-alpha tocopherol acetate preferably in an amount of 0.3 to 1 weight %; ferrous fumarate preferably in an amount of 0.2 to 1 weight %; folic acid preferably in an amount of 0.00 weight %; kale leaf preferably in an amount of 6 to 20 weight %; magnesium stearate preferably in an amount of 1 to 6 weight %; manganese sulfate preferably in an amount of 0.04 to 0.08 weight %; niacinamide preferably in an amount of 0.3 to 1 weight %; pantothenic acid preferably in an amount of 0.1 to 0.6 weight %; phytonadione preferably in an amount of 0.00 weight %; potassium iodine preferably in an amount of 0 weight %; pyridoxine HCl preferably in an amount of 0.03 to 0.1 weight %; riboflavin preferably in an amount of 0.02 to 0.1 weight %; sodium molybdate preferably in an amount of 0.00 weight %; sodium selenate preferably in an amount of 0.00 weight %; spinach (leaf, powder) preferably in an amount of 6 to 20 weight %; thiamine mononitrate preferably in an amount of 0.02 to 0.1 weight %; tomatoes (fruit powder, sun dried, granules) preferably in an amount of 6 to 20 weight %; tribasic calcium phosphate preferably in an amount of 0.5 to 2 weight %; and zinc oxide preferably in an amount of 0.2 to 0.8 weight %.

Table 3 provides an example of nutrient content for each day of the 5 day FMD regimen based on an average 180-200 lbs. person. It should be appreciated that practice within plus/minus 30 or 20 percent of the values indicated in Table 3 is contemplated, optionally truncated to 2 or 3 significant figures. Tables 4 and 5 provide exemplary meal plans for implementing the FMD.

TABLE 3
The nutrient content for each day of the 5 day FMD
regimen based on an average 180-200 lbs. person.

| | | | Day 2, 3, 4, 5 |
|---|---|---|---|
| | | | and any |
| | | | additional |
| | Unit | Day 1 | days* |
| Total Fat | (g) | 70 | 37.25 |
| % DV | | 90 | 47.75 |
| Protein | (g) | 23 | 15 |
| % DV | | 47 | 30, 5 |
| Total | (g) | 103 | 95.5 |
| Carbohydrate | | 37 | 35 |
| % DV | | | |
| Sugars | (g) | 28, 84 | 17.74 |
| Dietary Fiber | (g) | 31 | 20 |
| % DV | | 112 | 72.25 |
| VIT A | (IU) | 3.253 | 3.043 |
| % DV | | 361 | 314.5 |
| VIT C | (mg) | 113 | 61.5 |
| % DV | | 125 | 71 |
| Calcium | (mg) | 400 | 249.3 |
| % DV | | 31 | |
| Iron | (mg) | 12 | 7.25 |
| % DV | | 64 | 40.25 |
| Sodium | (mg) | 2.126 | 24.5 |
| % DV | | 92 | 75.75 |
| Potassium | (mg) | 1.127 | 830 |
| % DV | | 24 | 15 |

% DV indicates the percent of daily value based on a 2.000 kcal diet updated to 2020 regulations.
*Average values

TABLE 4
Meal Plan Variety 1

| | Day 1 | Day 2 | Day 3 | Day 4 | Day 5 |
|---|---|---|---|---|---|
| Breakfast | L-Bar Nut-based¹ | L-Bar Nut-based¹ | L-Bar Nut-based¹ | L-Bar Nut-based¹ | L-Bar Nut-based¹ |
| | Herbal Tea | Herbal Tea | Herbal Tea | Herbal Tea | Herbal Tea |
| | Algal Oil (2) | | | | Algal Oil |
| Lunch | Tomato | Mushroom | Tomato | Vegetable | Tomato |
| | Soup Mix | Soup Mix | Soup Mix | Soup Mix | Soup Mix |
| | NIR-1⁴ (2) ⁵ | NIR-1⁴ (1) ⁵ | NIR-1⁴ (1) ⁵ | NIR-1⁴ (1) ⁵ | NR-1⁴ (1) ⁵ |
| | Crackers | Olives | Crackers | Olives | Crackers |
| | Olives | | | | |
| Afternoon | Herbal Tea | Herbal Tea | Herbal Tea | Herbal Tea | Herbal Tea |
| | L-Bar Nut-based¹ | Olives | | Olives | |
| Dinner | Minestrone | Minestrone | Minestrone | Minestrone | Minestrone |
| | Soup Mix | with Quinoa | Soup Mix | with Quinoa | Soup Mix |
| | L-Bar Choco Crisp² | Soup Mix | | Soup Mix | |
| | | L-Bar Choco Crisp² | | L-Bar Choco Crisp² | |
| All over | | L-Drink³ | L-Drink³ | L-Drink³ | L-Drink³ |
| the day | | | | | |

¹An example of the first nutrition bar.
²An example of the second nutrition bar.
³Energy drink with glucose substitute
⁴Micronutrient supplement composition.
⁵ Values in parenthesis are the number of doses.

TABLE 5
Meal Plan Variety 2

| | Day 1 | Day 2 | Day 3 | Day 4 | Day 5 |
|---|---|---|---|---|---|
| Breakfast | L-Bar Nut-based¹ | L-Bar Nut-based¹ | L-Bar Nut-based¹ | L-Bar Nut-based¹ | L-Bar Nut-based¹ |
| | Herbal Tea | Herbal Tea | Herbal Tea | Herbal Tea | Herbal Tea |
| | Algal Oil (2) | | | | Algal Oil |
| Lunch | Butternut Squash | Tomato | Butternut Squash | Tomato | White Bean |
| | Soup Mix | Soup Mix | Soup Mix | Soup Mix | and Spinach |
| | NIR-1⁴ (2) ⁵ | NR-1 (1) ⁵ | NIR-1⁴ (1) ⁵ | NIR-1⁴ (1) ⁵ | Soup Mix |
| | Crackers | Olives | Crackers | Olives | NIR-1⁴ (1) ⁵ |
| | Olives | | | | Crackers |
| Afternoon | Herbal Tea | Herbal Tea | Herbal Tea | Herbal Tea | Herbal Tea |
| | L-Bar Nut-based¹ | Olives | | Olives | |
| Dinner | Black Bean | Butternut Squash | White Bean | Black Bean | Butternut Squash |
| | Soup Mix | and Quinoa | and Spinach | Soup Mix | and Quinoa |
| | L-Bar Choco Crisp² | Soup Mix | Soup Mix | L-Bar Choco Crisp | Soup Mix |
| | | L-Bar Choco Crisp² | | | |
| All over | | L-Drink³ | L-Drink³ | L-Drink³ | L-Drink³ |
| the day | | | | | |

¹An example of the first nutrition bar.
²An example of the second nutrition bar.
³Energy drink with glucose substitute
⁴Micronutrient supplement composition.
⁵ Values in parenthesis are the number of doses.

The micronutrient supplement composition in these meal plans is a vegetable powder with vitamins and minerals supplements. With respect to the 5 day meal plans, it should be appreciated that it the diet is administered for fewer days, the identified protocol for a given day is used. For example, for a 3 day FMD, days 1, 2, and 3 are used. Likewise, for a FMD that is administered for greater than 5 days, any of the daily protocols for days 2-5 can be used for these additional days.

In some variations, a refeeding diet is administered to the subject for a second time period. The refeeding diet provides an overall calorie consumption that is within 10 percent of a subject's normal calorie consumption. In this context, normal calorie consumption is the number of calories needed to maintain the subject's weight at their pretreatment weight or to maintain a predetermined target weight. Therefore, the refeeding diet can be referred to as a “refeeding diet.” Although the present invention is not significantly limited by the second time period, the second time period can be from 7 days to 6 months or longer. Typically, the second time period is from 5 days to 26 days or longer following the FMD. In some refinements, the refeeding diet provides at most, in increasing order of preference, 2500 kcal/day, 2400 kcal/day, 2300 kcal/day, 2200 kcal/day, 2100 kcal/day, 2000 kcal/day, 1900 kcal/day, 1800 kcal/day, 1700 kcal/day, 1600 kcal/day, or 1500 kcal/day. In some further refinements, the refeeding diet provides at least, in increasing order of preference, 1200 kcal/day, 1300 kcal/day, 1400 kcal/day, 1500 kcal/day, 1600 kcal/day, 1700 kcal/day, or 1800 kcal/day.

In a variation, the cycle of an FMD for a first time period and the refeeding diet for a second time period is repeated a plurality of times over a plurality of months or years. In a refinement, the cycle is repeated for 1 to 25 cycles.

In another embodiment, an immune-enhancing ketogenic fasting-mimicking diet (FMD) package for improving immune profile and function is provided. Advantageously, compositions of the package are portioned to attain fasting-like effects on serum levels of IGF-1, IGFBL1, glucose, and ketone bodies while providing both macronutrients and micronutrients to minimize the burden of fasting (e.g., no calories for a one or more days) and adverse effects of fasting. The immune-enhancing ketogenic fasting-mimicking diet package includes breakfast, lunch, and dinner meal portions for administering an immune-enhancing ketogenic fasting-mimicking diet for each day of a first time period. In particular, the immune-enhancing ketogenic fasting-mimicking diet package includes nutrition bars and soups to be consumed for breakfast, lunch, and dinner as set forth above. In a refinement, the immune-enhancing ketogenic fasting-mimicking diet is administered for a first time period of 1, 2, 3, 4, 5, 6, 7, 8, 9, or 10 days. Typically, these are consecutive days.

The immune-enhancing ketogenic fasting-mimicking diet package provides rations for implementing the immune-enhancing ketogenic fasting-mimicking diet. The FMD is formulated to provide 800 to 1250 kcal on day 1 and 500 to 950 kcal on days 2-5 or any additional days. In a variation, the immune-enhancing ketogenic fasting-mimicking diet package includes rations that provide 2.4 to 7 kcal per day for each day of the immune-enhancing ketogenic fasting-mimicking diet, less than or equal to 30 g of sugar for each day of the immune-enhancing ketogenic fasting-mimicking diet, less than 30 g of proteins for each day of the immune-enhancing ketogenic fasting-mimicking diet, 15-30 grams of monounsaturated fats for each day of the immune-enhancing ketogenic fasting-mimicking diet, 2-10 g of polyunsaturated fats for each day of the immune-enhancing ketogenic fasting-mimicking diet, and less than 12 g of saturated fats for each day of the immune-enhancing ketogenic fasting-mimicking diet.

In a variation, the immune-enhancing ketogenic fasting-mimicking diet package includes rations for day 1 that provide 3.5 to 5 kcal per pound of the subject. In a refinement, the immune-enhancing ketogenic fasting-mimicking diet package includes rations for day 1 that provide less than 25 g of sugar, less than 23 g of proteins, 16-25 grams of monounsaturated fats, 4.8 to 8 g of polyunsaturated fats, and 1 to 10 g of saturated fats. In this variation, the immune-enhancing ketogenic fasting-mimicking diet package includes rations for days 2 to 5 or any additional days, total calorie consumption that provides 2.4 to 4 kcal per pound of the subject for each day. In a refinement, the immune-enhancing ketogenic fasting-mimicking diet package includes rations days 2 to 5 or any additional days that provide less than 16 g of sugars, less than 15 g of protein, 8 to 12 g of monounsaturated fats, 2 to 4 g of polyunsaturated fats and 1 to 6 grams of saturated fats.

In another variation, the immune-enhancing ketogenic fasting-mimicking diet packages include rations for the soups, nutrition bars, crackers, an optional drink with a glucose substitute, olives, vitamin and mineral supplements, algal oil, and optional teas as set forth above. Therefore, the details of these components for the immune-enhancing ketogenic fasting-mimicking diet packages are the same as set forth above.

In another embodiment, a method for preparing the immune-enhancing ketogenic fasting-mimicking diet package set forth above is provided. The method includes a step of receiving subject information for a subject in need of or desiring improving immune profile and function that includes a subject's weight and optionally their age. In a refinement, the subject information also includes the medical history (as set forth above), gender, and diagnosis of the subject. Meal portions are formed for each day of a predetermined time period during which an immune-enhancing ketogenic fasting-mimicking diet is administered. These meal portions can include a breakfast portion, a lunch portion, and a dinner portion. In a refinement, the meal portions further include snack portions and drink portions. Characteristically, the meal portions provide a subject with at most 50 percent of a subject's normal calorie consumption per day with protein and sugar rejection as set forth above. In a refinement, the diet package is assembled to provide from 30 to 50 percent of the average normal calorie consumption per day with protein and sugar rejection as set forth above. In a refinement, the normal caloric consumption per day can be defined by the following formula:

$$normal caloric intake = \frac{2000 kcal}{180 lb} \times ( {subject}^{′} s weight in pounds ) .$$

In another variation, subject information for a plurality of subjects in need of or desiring improving immune profile and function is received. The plurality of subjects can be grouped with predetermined weight ranges, each range having an average weight. Diet packages for each weight range are assembled based on the average weight for each weight range. For example, all subjects having a normal weight below 200 can be treated with the unscaled FMD while heavier subjects treated with a scaled version. In a refinement, the weight ranges can incremented from a start weight with a predefined value (with each range including only the upper or lower endpoint). For example, the weight ranges can be 100-120 lbs., 100-120 lbs., 120-140 lbs., 140-160 lbs., 160-180 lbs., 180-200 lbs., 200-220 lbs., 220-240 lbs., 240-260 lbs. etc. Useful increments can be 5 lbs., 10 lbs., 15 lbs., 20 lbs., 25 lbs. etc. The diet packages are assembled to provide at most 50% of an average normal caloric consumption per day for each weight range with protein and sugar restriction as set forth above. In a refinement, the diet packages are assembled to provide a from 30 to 50 percent of the average normal calorie consumption per day with protein and sugar restriction as set forth above. In a variation, the average normal caloric consumption per day for a weight range is given by:

$$average normal caloric intake = \frac{2000 kcal}{180 lb} \times ( middpoint of a weight range ) .$$

It should be appreciated that in these variations for preparing diet package(s), the ranges and amounts for calorie intake, fat, carbohydrates, protein, sugars as well as the compositions for the soups, nutrition bars and other FMD components can be independently scaled by a factor (e.g., subject's weight in pounds divided by 180 lbs. or midpoint of a weight range divided by 180 lbs.) that adjusts the amounts to compensate for a subject's weight. For example, the immune-enhancing ketogenic fasting-mimicking diet and the immune-enhancing ketogenic fasting-mimicking diet package that provides 2.4 to 7 kcal per day for each day of the immune-enhancing ketogenic fasting-mimicking diet, less than or equal to 30 g of sugar for each day of the immune-enhancing ketogenic fasting-mimicking diet, less than 30 g of proteins for each day of the immune-enhancing ketogenic fasting-mimicking diet, 15-30 grams of monounsaturated fats for each day of the immune-enhancing ketogenic fasting-mimicking diet, 2-10 g of polyunsaturated fats for each day of the immune-enhancing ketogenic fasting-mimicking diet, and less than 12 g of saturated fats for each day of the immune-enhancing ketogenic fasting-mimicking diet can be scaled by a factor of subjects weight in pounds divided by 180 lbs.

It should also be appreciated that diet packages can be prefabricated before being requested or receiving the subject information. In this case, upon receiving the subject information or request for an order for a weight or weight range, the diet package is sent to a customer (e.g., consumer).

The following examples illustrate the various embodiments of the present invention. Those skilled in the art will recognize many variations that are within the spirit of the present invention and scope of the claims.

The results set forth below show that a new immune-enhancing FMD formulation tested in aged mice induced an activation of white blood cells, lymphocytes, and hematopoietic stem cells in comparison of control fed mice. The competitive repopulation assay showed, compared to the control fed group, bone marrow (BM) cells from mice subjected to the immune-enhancing FMD have higher regeneration capacity leading to efficient blood reconstitution and improved engraftment in the blood and BM. These results strongly support a role for FMD diets in stimulation of HSC self-renewal and improvement in both short- and long-term hematopoietic repopulation capacities. Furthermore, the B-cell population was increased indicating an improved potential for immune response capacity. Secondary transplantation further confirmed the beneficial effects of our FMD in long-term repopulation capacity of BM cells. The immune-enhancing FMD showed significant increases in antibody titers against influenza virus strains, A(H3N2) and B(Victoria) in female mice. The formulation of the FMD identified to be effective in mice will be developed for human purpose.

Methods

Experimental Approach: Male and female 18-month-old mice were enrolled in either control or diet arms (N=10/group). The diet group is exposed to 2 dietary intervention cycles, each followed by a 21-day refeeding period. All animal protocols were approved by the Institutional Animal Care and Use Committee (IACUC) of the University of Southern California. All mice were maintained in a pathogen-free environment and housed in clear shoebox cages in groups of five animals per cage with constant temperature and humidity and 12 hr/12 hr light/dark cycle and unlimited access to water.

Diets: The immune-enhancing FMD is a low-calorie diet enriched with coconut oil based on human foods. The source of calories is 32% from carbohydrates, 6% protein and 62% fat. The diet is mainly composed by nut bars, olives, kale crackers, vegetable soup, pumpkin soup, mushroom soup, tomato soup. The breakdown of fat sources is approximately 47% from coconut oil; ˜36% of fat (by weight) is derived from nuts that are part of the energy bars (almond meal, macadamia nut butter, Pecan, Coconut, flaxseed meal and coconut oil); ˜10% of from flax seeds, sunflower seeds, cashews, sesame seeds that are ingredients of the crackers; ˜5% from vegetables (soups); and ˜3% from olives. 54% of the fat is saturated fat (primarily from coconut oil which is rich in medium chain fatty acids). All diet ingredients were thoroughly mixed and then blended together with heated hydrogel (ClearH2O, Maine).

Mice were fed at 70% normal daily intake on day 1 and 50% normal daily intake on day 2-5. Flu vaccination. 2 cycles of the immune-enhancing FMD were performed in 3 mo and 18 mo male and female C57BL/6 mice (N=10/group). Tail vein blood (25 ul) were sampled prior to enrollment, at the end of the first cycle and one week after re-feeding on the last cycle to confirm effects on glucose, and blood cell counts (CBC). At the end of the 2nd cycle mice were injected with split virus vaccine (Sanofi Pasteur, Swiftwater, Pa.)(15 ug HA, i.m.). Tail blood samples were collected weekly for testing in a standard hemagglutination inhibition assay to assess the B-cell antibody response. 56 days after vaccination, mice were euthanized, and cardiac blood were collected for further testing.

Hematopoietic stem cells (HSC) were characterized performing FACS analysis of Short Term, Long Term and MPP HSC. Briefly bone marrow cells were harvested from the femur and tibia of mice and stained with lineage-specific Lin marker, Scal-1 (PE-Cy-A) and c-Kit (APC-A) antibodies to measure LSK population Long-term (LT-HSC) and short-term (ST-HSC) hematopoietic stem cells (HSCs) as well as the multipotent progenitors (MPPs) will be characterized by FACS based on the expression of SLAM family markers, CD150, CD48, in the Lin-Sca-1+c-Kit+ cells. To also detect cell genesis, cell were stained with Ki67 antibody to detect the proliferative compartment

Transplantation experiment: Following 2 cycles of FMD, bone marrow mononuclear cells (BMMCs) (2.5×10⁵) were harvested from 18 mo old C57BL/6 mice (N=10/group) and mixed with an equal number of BMMCs from competitor mouse (B6.SJL). These cells were injected into recipient B6.SJL, lethally irradiated 24 h previously with 10 Gy of radiation. Total cell counts were assessed by differential CBC. The relative contribution of engraftment from the different cell sources was assessed by flow cytometry for the reconstituted peripheral blood cells with CD45.2 (C57B/6) and CD45.1 (B6.SJL) antigens.

Second transplant: Following 4 months after the first competitive transplantation, bone marrow mononuclear cells (BMMCs) (3×10⁶) were harvested from C57BL/6N mice (N=6/group) and injected into new recipient B6. SJL mice, lethally irradiated 24 h previously with 10 Gy of radiation. The relative contribution of engraftment from the different cell sources was assessed by flow cytometry for the reconstituted peripheral blood cells with CD45.2 (C57B/6) and CD45.1 (B6. SJL) antigens.

Lymphoid: Myeloid ratios in human subjects: In brief, one-hundred participants (generally healthy adult volunteers and 18 to 70 years of age; BMI, 18.5 and up) without a diagnosed medical condition in the previous 6 months were enrolled in Trial NCT02158897. Participants completed three cycles of this 5-day FMD. Participants completed baseline (BL) and follow-up examinations at the end of the first FMD (before resuming normal diet to measure the acute FMD effects; 1 FMD) and after a washout period of 5 to 7 days of normal caloric intake after the third FMD cycle (3 FMD). Blood draw was completed at each visit and complete blood counts were immediately determined.

Statistics and experimental design: All samples represent biological replicates. Unless otherwise specified in figure legends, all center values shown in graphs refer to the mean. For statistical significance of the differences between the means of two groups, we used two-tailed Student's t-tests. Statistical significance of differences among multiple groups (>3) was calculated by performing ANOVA multiple comparisons of the means for each group.

No samples or animals were excluded from analysis, and sample size estimates were not used. Animals were randomly assigned to groups. Studies were not conducted blinded.

Results:

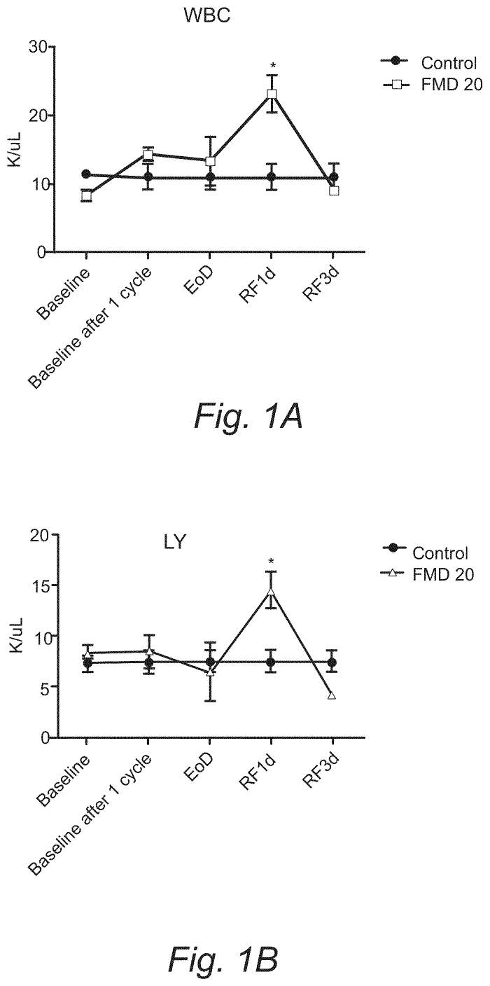

We show results from old 18 month mice subjected to 2 cycles of the immune-enhancing FMD, and ad libitum (control) feeding. We collected white blood cells (WBC) and lymphocytes (LY) at the baseline, after one cycle, at the end of the diet (EoD), after refeeding for 1 day (RF1d), and after refeeding for 3 days (RF3d). The dietary intervention FMD20 induced an increase of the total number of WBCs and LYs at RF1d followed by a return to normal levels at the end of the three days of re-feeding, indicating an activation of the white blood cells during the refeeding time (FIG. 1).

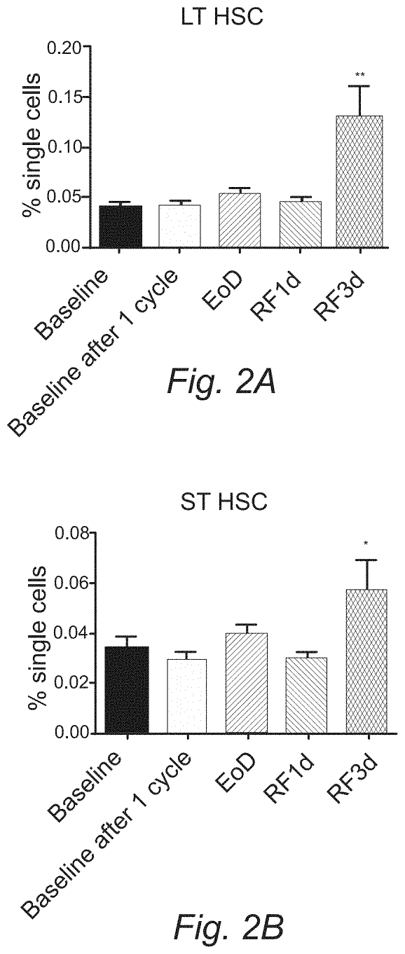

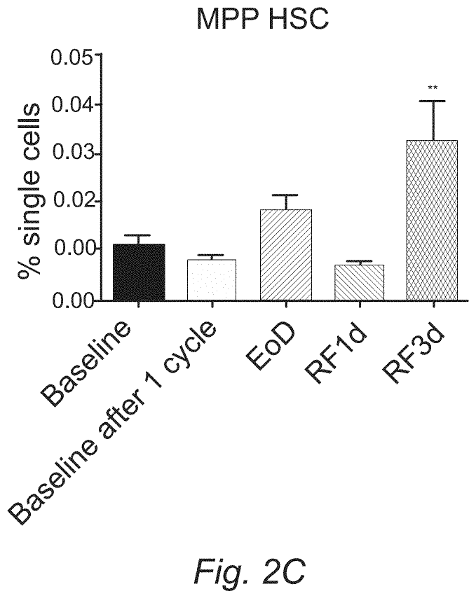

Bone marrow (BM) was collected at the 4 different time points after 2 cycles of dietary intervention. Bone marrow cells were assayed by flow cytometry for long-term (LT-HSC) and short-term (ST-HSC) hematopoietic stem cells (HSCs) as well as the multipotent progenitors (MPPs) based on the expression of SLAM family markers, CD150, CD48, in the lineage-/low Sca-1+c-Kit+ cells (FIG. 2). After refeeding for 3 days all three hematopoietic stem cell population increased.

The immune-enhancing FMD Bone marrow Functionality Results: 18 month C57BL/6N mice were subjected to 2 cycles of the immune-enhancing FMD, and ad libitum (control) feeding. At RF1 day bone marrow mononuclear cells (BMMCs) were harvested from 18 mo old C57BL/6N mice and mixed with an equal number of BMMCs from competitor mouse (B6. SJL) and transplanted in irradiated recipient B6. SJL (8 weeks old) (N=6). After 4, 8, 12 and 16 weeks from transplantation the relative contribution of engraftment from the different cell sources were assessed by flow cytometry for the reconstituted peripheral blood cells with CD45.2 (B6) and CD45.1 (B6. SJL) antigens.

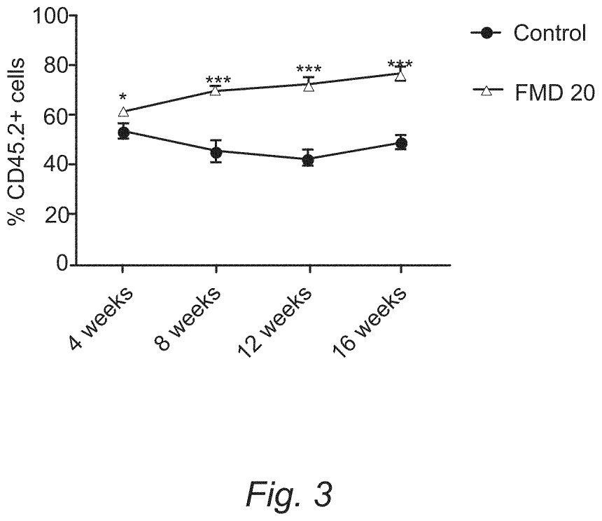

4 weeks after transplantation in the peripheral blood of male mice the percentage of CD45.2+ cells was higher in the recipient mice that received the bone marrow transplant from donor mice that underwent to two dietary cycles than control mice (FIG. 3). Data confirmed also after 8, 12 and 16 weeks from transplant.

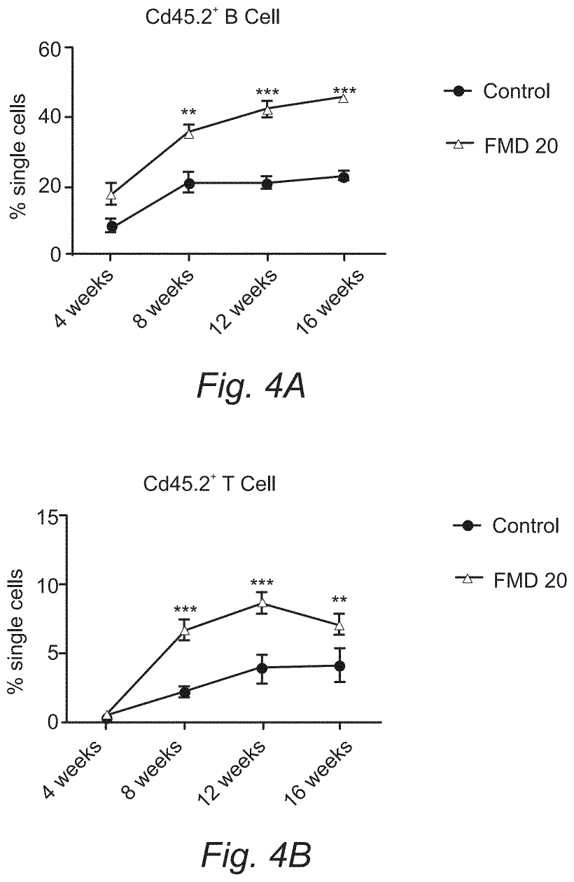

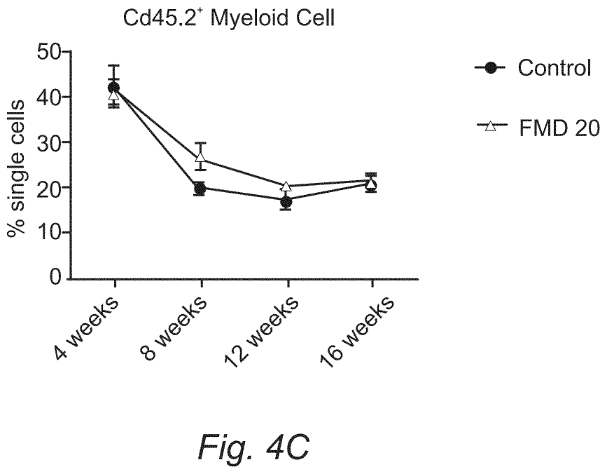

The lymphoid population was assessed by flow cytometry of the RF1D recipient peripheral blood (PB) with B220+(B cell), CD3+(T cell) or Mac1 & GR1 (myeloid cell) staining (FIG. 4). B cells and similarly, T cells were increased, myeloid cell population unchanged.

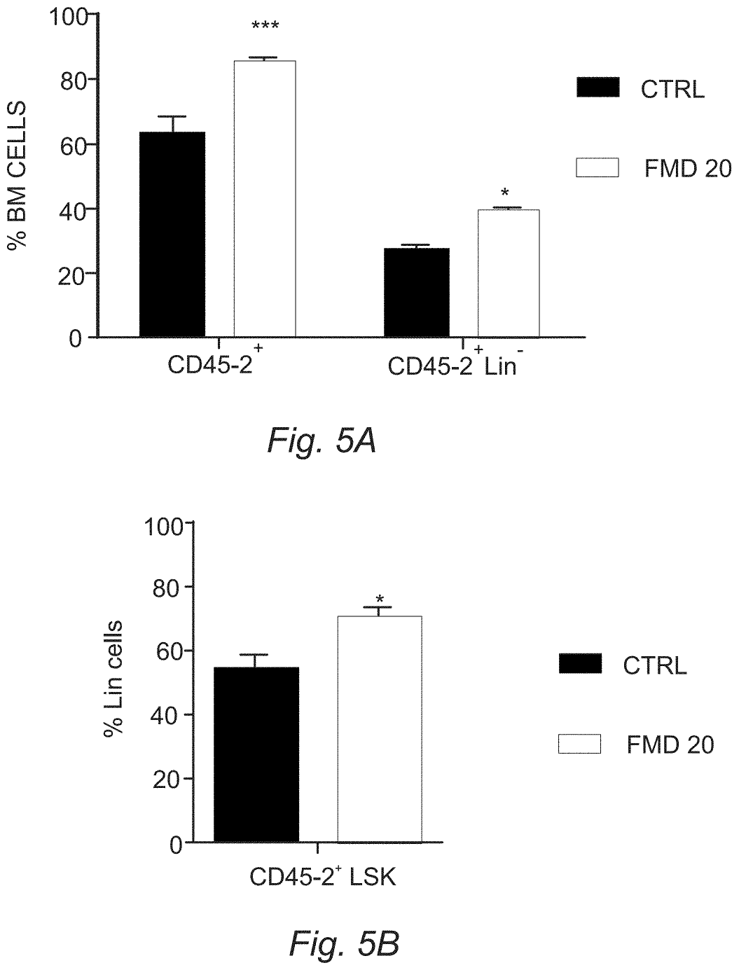

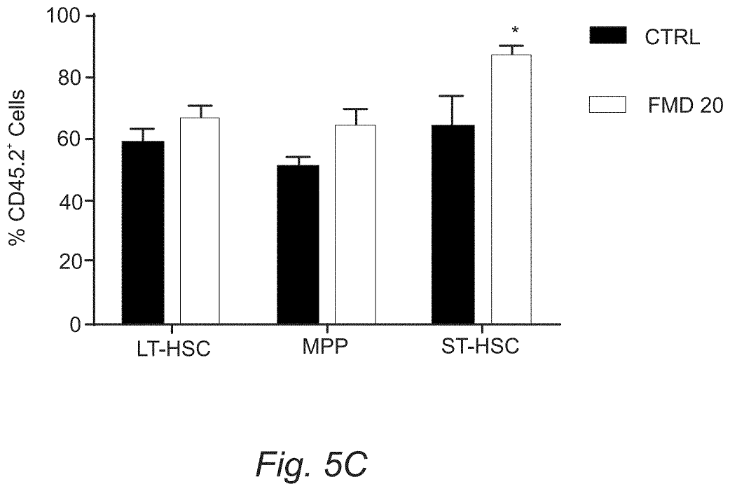

Bone marrow (BM) was collected after 4 months. CD45.2+ bone marrow cells were assayed by flow cytometry for long-term (LT-HSC) and short-term (ST-HSC) hematopoietic stem cells (HSCs) as well as the multipotent progenitors (MPPs) based on the expression of SLAM family markers, CD150, CD48, in the lineage-/low Sca-1+c-Kit+ cells (FIG. 5).

Bone marrow analyses confirmed the higher percentage of CD45.2+ cells in recipient mice that received the bone marrow transplant from the RF1D donor mice than control. Mice showed also a significant increase of LT-HSC, ST-HSC.

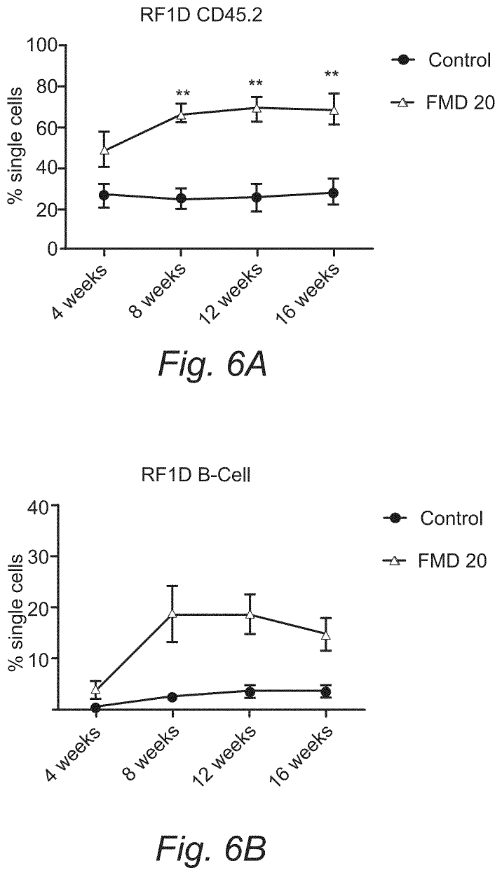

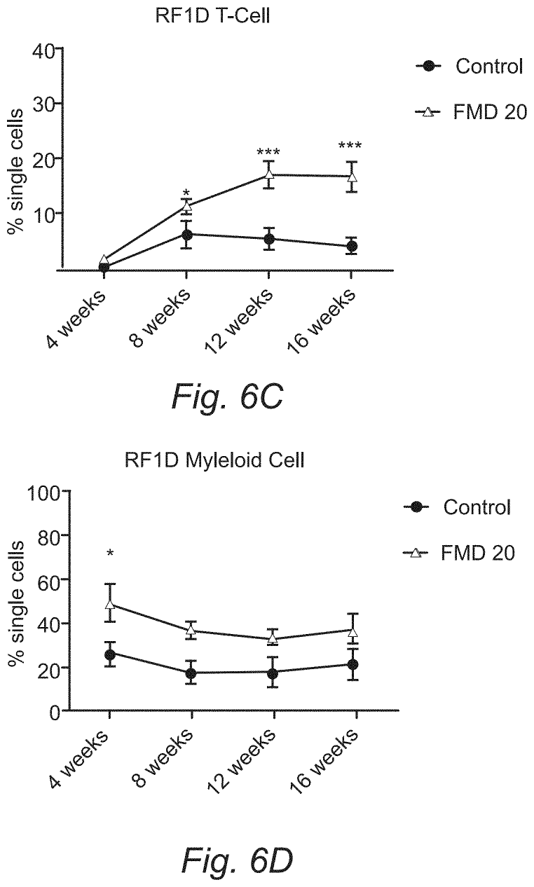

To test the functionality of bone marrow cells from mice that were subjected to cycles of FMD we performed a second transplant. Following 4 months after the first competitive transplantation, bone marrow mononuclear cells (BMMCs) (3×10⁶) were harvested from C57BL/6N mice (N=6/group) and injected into new recipient B6.SJL mice, lethally irradiated 24 h previously with 10 Gy of radiation. The relative contribution of engraftment from the different cell sources was assessed by flow cytometry for the reconstituted peripheral blood cells with CD45.2 (C57B/6) and CD45.1 (B6.SJL) antigens both short (4 and 8 weeks) and long-term (12 and 16 weeks) engraftment. After 8 weeks from second transplantation in the peripheral blood of male mice the percentage of CD45.2+ cells was higher in the immune-enhancing FMD (FIG. 6). Data confirmed also after 12 and 16 weeks from transplant. The immune-enhancing FMD increased T cells population and a positive trend in B and myeloid compartment was also observed.

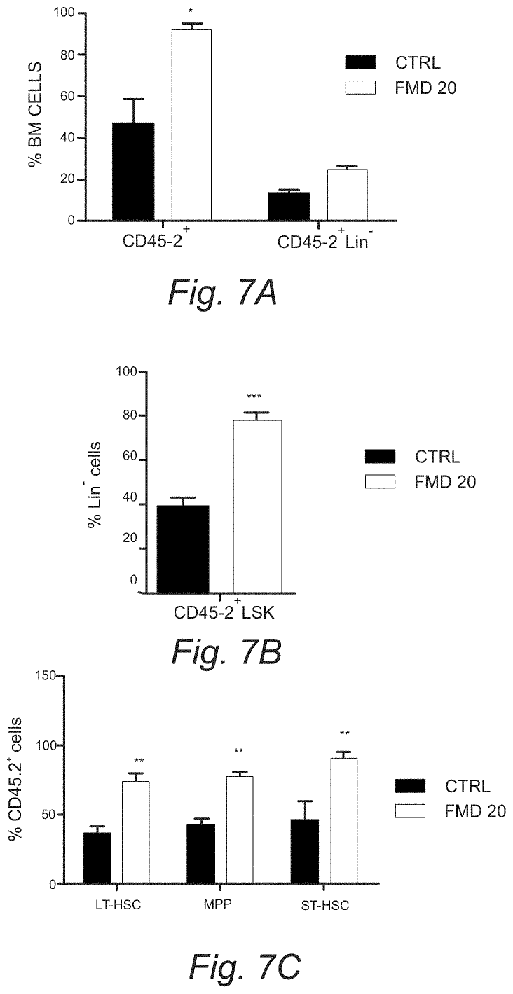

Bone marrow (BM) was collected after 4 months following the second transplant. CD45.2+ bone marrow cells were assayed by flow cytometry for long-term (LT-HSC) and short-term (ST-HSC) hematopoietic stem cells (HSCs) as well as the multipotent progenitors (MPPs) based on the expression of SLAM family markers, CD150, CD48, in the lineage-/low Sca-1+c-Kit+ cells (FIG. 7). Bone marrow analyses of male and female mice confirmed the higher percentage of CD45.2+ cells in the immune-enhancing FMD than control. Both sexes showed also an increase of LT-HSC, ST-HSC & MPPs.

Vaccination Results: 18 month and 3 month male and female mice were subjected to 2 cycles of the immune-enhancing FMD, and ad libitum (control) feeding. The vaccination response was analyzed 28 days after vaccination by the hemagglutination inhibition assay (HAI). The titer of specific antibodies in 18 mo female mice fed with the immune-enhancing FMD was increased.

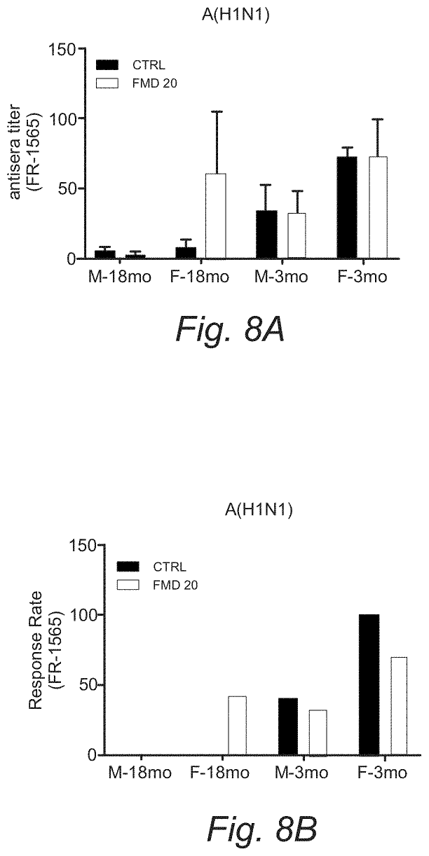

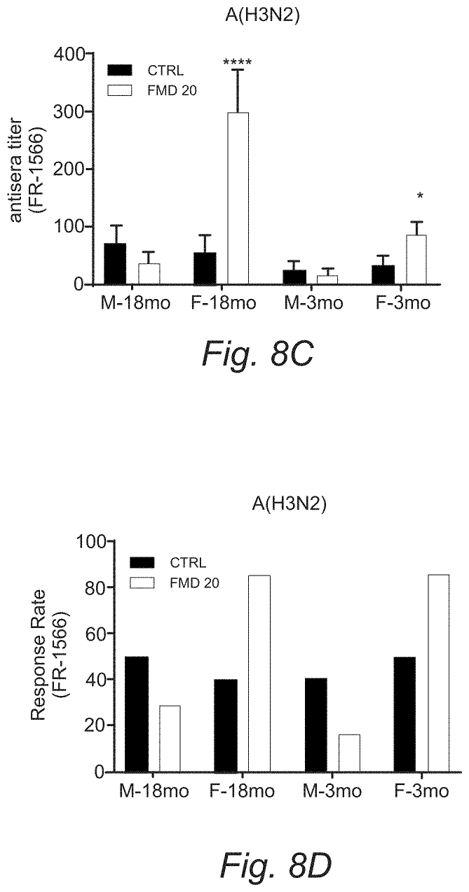

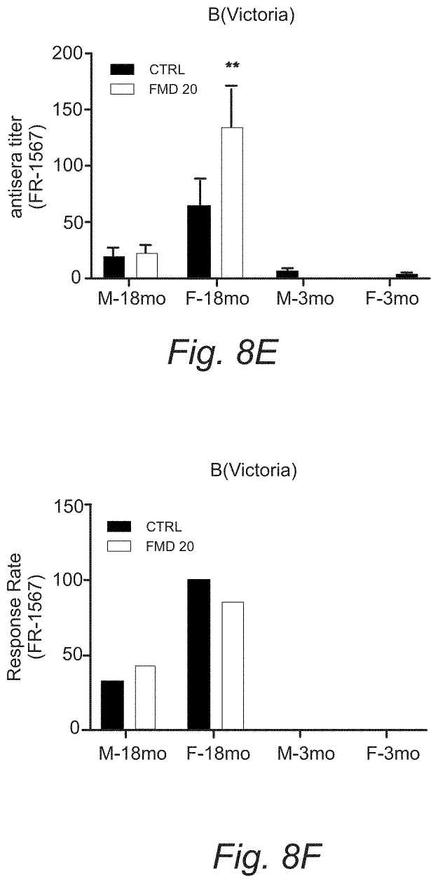

Specifically, after 28 days (FIG. 8), 18 mo females on FMD 20 showed significant increases in the titer of antibody against strains, A(H3N2) (FIG. 8C) and B(Victoria) (FIG. 8E).

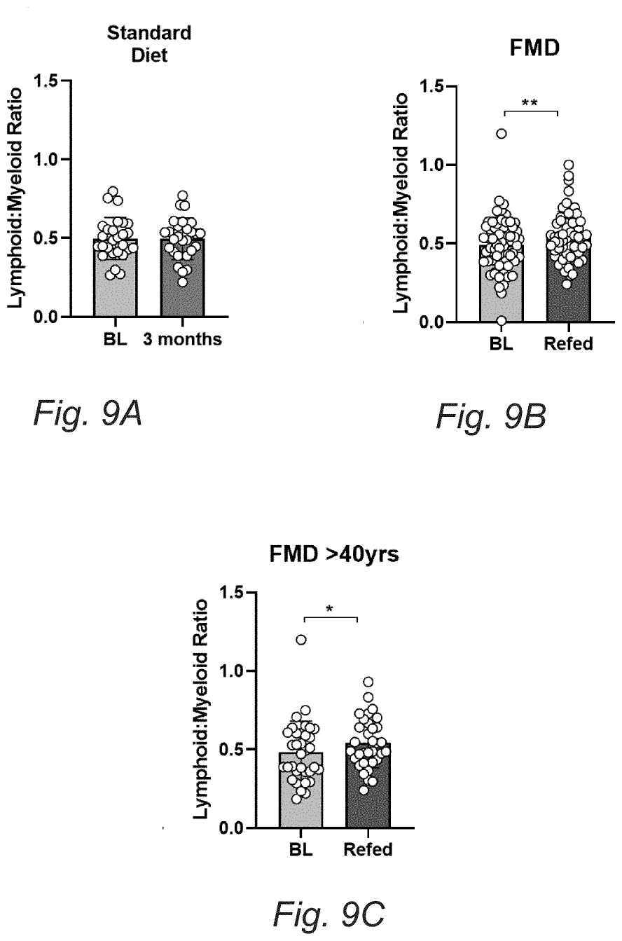

Immune rejuvenation in human subjects: The Lymphoid/Myeloid ratio (L/M) did not change in subjects that maintained a regular diet (FIG. 9A, N=27). An improvement in the L/M ratio was measured in all FMD subjects (FIG. 9B, N=61) and particularly in the over 40-year-old subjects (FIG. 9C, N=34) following 3 ketogenic FMD cycles and after returning to the normal diet. Thus, 3 FMD cycles in humans, promote changes consistent with previous results in mice indicating rejuvenating effects on the immune system.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Raffaghello, L., et al. Starvation-dependent differential stress resistance protects normal but not cancer cells against high-dose chemotherapy. Proc Natl Acad Sci USA 105, 8215-8220 (2008).
2. Lee, C., et al. Fasting cycles retard growth of tumors and sensitize a range of cancer cell types to chemotherapy. Sci Transl Med 4, 124ra127 (2012).
3. Choi, I. Y., et al. A Diet Mimicking Fasting Promotes Regeneration and Reduces Autoimmunity and Multiple Sclerosis Symptoms. Cell Rep 15, 2136-2146 (2016).
4. Brandhorst, S., et al. A Periodic Diet that Mimics Fasting Promotes Multi-System Regeneration, Enhanced Cognitive Performance, and Healthspan. Cell Metab 22, 86-99 (2015).
5. Cheng, C. W., et al. Prolonged fasting reduces IGF-1/PKA to promote hematopoietic-stem-cell-based regeneration and reverse immunosuppression. Cell Stem Cell 14, 810-823 (2014).
6. McElhaney, J. E. & Effros, R. B. Immunosenescence: what does it mean to health outcomes in older adults? Curr Opin Immunol 21, 418-424 (2009).
7. McElhaney, J. E., et al. The unmet need in the elderly: how immunosenescence, CMV infection, co-morbidities and frailty are a challenge for the development of more effective influenza vaccines. Vaccine 30, 2060-2067 (2012).
8. Chou, J. P. & Effros, R. B. T cell replicative senescence in human aging. Curr Pharm Des 19, 1680-1698 (2013).
9. Panda, A., et al. Human innate immunosenescence: causes and consequences for immunity in old age. Trends Immunol 30, 325-333 (2009).
10. Frasca, D., et al. Aging down-regulates the transcription factor E2A, activation-induced cytidine deaminase, and Ig class switch in human B cells. J Immunol 180, 5283-5290 (2008).
11. Frasca, D., Landin, A. M., Riley, R. L. & Blomberg, B. B. Mechanisms for decreased function of B cells in aged mice and humans. J Immunol 180, 2741-2746 (2008)

What is claimed is:

1. A method for improving immune profile and function comprising:
providing or administering an immune-enhancing ketogenic fasting-mimicking diet to a subject in need of immune profile and function improvement.

2. The method of claim 1, wherein the subject is greater than or equal to 50 years old.

3. The method of claim 1, wherein the subject is greater than or equal to 60 years old.

4. The method of claim 1, wherein the subject is greater than or equal to 70 years old.

5. The method of claim 1, wherein the immune-enhancing ketogenic fasting-mimicking diet is administered for a first time period of 1, 2, 3, 4, 5, 6, 7, 8, 9, or 10 days.

6. The method of claim 1, wherein the immune-enhancing ketogenic fasting-mimicking diet is administered 3 to 6 months before flu season.

7. The method of claim 1, wherein the immune-enhancing ketogenic fasting-mimicking diet provides 750 to 1250 kcal for day 1 and 1100 to 1500 kcal for days 2-5 and any additional days.

8. The method of claim 1, wherein the immune-enhancing ketogenic fasting-mimicking diet provides 850 to 1150 kcal for day 1 and 1200 to 1400 kcal for days 2-5 and any additional days.

9. The method of claim 1, wherein the immune-enhancing ketogenic fasting-mimicking diet is formulated to provide 20 to 40% calories from carbohydrates, 2 to 12% calories from protein, and 50 to 75% calories from fat.

10. The method of claim 1, wherein the immune-enhancing ketogenic fasting-mimicking diet includes nutrition bars and soups to be consumed for breakfast, lunch, and dinner.

11. The method of claim 10, wherein the immune-enhancing ketogenic fasting-mimicking diet includes an FMD soup formulated to provide less than about 1 gram of saturated fat per serving, less than about 5 grams of trans fat per serving, less than about 10 grams of protein per serving, less than about 40 grams of carbohydrates per serving.

12. The method of claim 11, wherein the FMD soup is formulated from any combination of components selected from the group consisting of black beans, butternut squash, quinoa, tomatoes, mushrooms, white beans, brown beans, spinach, green tea extract, rice flour, onions, brown rice powder, carrots, inulin, leeks, olive oil, cabbage, potatoes, olives, peas, pumpkin, maltodextrin, and celery, chicory root fiber, sea salt, yeast, basil, parsley, garlic, rosemary extract, coriander, oregano, potato starch, potato flakes, zucchini squash, turmeric.

13. The method of claim 11, wherein the FMD soup is formulated from a first component selected from the group consisting of black beans, butternut squash, quinoa, tomatoes, mushrooms, white beans, brown beans, spinach, and combinations thereof and a second component selected from the group consisting of green tea extract, rice flour, onions, brown rice powder, carrots, inulin, leeks, olive oil, cabbage, potatoes, olives, peas, pumpkin, maltodextrin, and celery, chicory root fiber, sea salt, yeast, basil, parsley, garlic, rosemary extract, coriander, oregano, potato starch, potato flakes, zucchini squash, turmeric, and combinations thereof.

14. The method of claim 10, wherein the immune-enhancing ketogenic fasting-mimicking diet includes a first FMD nutrition bar formulated to provide about bout 150 kcal to 350 kcal per serving, 10 to 35 grams of total fat per serving, less than about 8 grams of saturated fat per serving, less than about 5 grams of trans fat per serving, less than about 10 grams of protein per serving, and less than about 30 grams of carbohydrates per serving.

15. The method of claim 14, wherein the first FMD nutrition bar is formulated from a combination of macadamia nuts, honey, pecans, almonds, almond butter, coconut flour, sea salt, mixed tocopherols (vitamin E), citric acid, and/or ascorbic acid.

16. The method of claim 10, wherein the immune-enhancing ketogenic fasting-mimicking diet includes a second FMD nutrition bar formulated to provide about bout 70 kcal to 120 kcal per serving, 3 to 10 grams of total fat per serving, less than about 5 grams of saturated fat per serving, less than about 5 gram of trans fat per serving, less than about 6 grams of protein per serving, and less than about 20 grams of carbohydrates per serving.

17. The method of claim 16, wherein the second FMD nutrition bar is formulated from a combination of inulin, almond butter, brown rice crispy, cocoa powder, almonds, chocolate chips, rolled oats, brown rice syrup, flaxseed oil, rice dextrin, grape juice, and salt.

18. The method of claim 1, wherein the immune-enhancing ketogenic fasting-mimicking diet includes a drink that includes a glucose substitute.

19. The method of claim 1, wherein the immune-enhancing ketogenic fasting-mimicking diet includes nut bars, olives, kale crackers, vegetable soup, pumpkin soup, mushroom soup, tomato soup.

20. The method of claim 19, wherein a breakdown of fat sources in the immune-enhancing ketogenic fasting-mimicking diet is approximately 35 to 50 weight % from coconut oil; 25 to 45 weight derived from nuts within nutrition bars; 5 to 25 weight % from flax seeds, sunflower seeds, cashews, sesame seeds that are ingredients of the kale crackers; about 2 10 weight % from vegetables; and about 1 to 6 weight percent from olives.

21. The method of claim 20, wherein the immune-enhancing ketogenic fasting-mimicking diet is formulated such that from 45 to 60 weight % of fats provided is saturated fat.

22. The method of claim 1, wherein a refeeding diet is administered to the subject for a second time period.

23. The method of claim 22, wherein the immune-enhancing ketogenic fasting-mimicking diet and the refeeding diet are repeated for a plurality of cycles.

24. A method for preparing an immune-enhancing ketogenic fasting-mimicking diet package for treating a subject that is in need of improving immune profile and function, the immune-enhancing ketogenic fasting-mimicking diet package including breakfast, lunch, and dinner meal portions for administering an immune-enhancing ketogenic fasting-mimicking diet for each day of a first time period. the method comprising:
receiving subject information for the subject that includes a subject's weight; and
forming meal portions for each day of a predetermined time period during which the immune-enhancing ketogenic fasting-mimicking diet is administered, the meal portions providing the subject with at most 80 percent of a subject's normal calorie consumption per day with protein and sugar rejection.

25. The method of claim 24, wherein the immune-enhancing ketogenic fasting-mimicking diet is formulated to provide 2.0 to 12 kcal per day for each day of the immune-enhancing ketogenic fasting-mimicking diet, less than or equal to 30 g of sugar for each day of the immune-enhancing ketogenic fasting-mimicking diet, less than 30 g of proteins for each day of the immune-enhancing ketogenic fasting-mimicking diet, 15-30 grams of monounsaturated fats for each day of the immune-enhancing ketogenic fasting-mimicking diet, 2-10 g of polyunsaturated fats for each day of the immune-enhancing ketogenic fasting-mimicking diet, and less than 12 g of saturated fats for each day of the immune-enhancing ketogenic fasting-mimicking diet.

26. The method of claim 25, wherein the immune-enhancing ketogenic fasting-mimicking diet is formulated to provide 2.4 to 7 kcal per day for each day of the immune-enhancing ketogenic fasting-mimicking diet.

27. The method of claim 25, wherein the ranges are scaled with by a factor of subjects weight in pounds divided by 180 lbs.

28. The method of claim 24, wherein the meal portions provide the subject with at most 50 percent of the subject's normal calorie consumption per day.

29. The method of claim 24, wherein the immune-enhancing ketogenic fasting-mimicking diet is administered for 1, 2, 3, 4, 5, 6, 7, 8, 9, or 10 consecutive days.

30. The method of claim 29, wherein the immune-enhancing ketogenic fasting-mimicking diet package includes rations formulated to provide 800 to 1250 kcal on day 1 and 500 to 950 kcal on days 2, 3, 4, 5, or any additional days.

31. The method of claim 29, wherein the immune-enhancing ketogenic fasting-mimicking diet package includes rations formulated to provide 2.4 to 7 kcal per day for each day of the immune-enhancing ketogenic fasting-mimicking diet, less than or equal to 30 g of sugar for each day of the immune-enhancing ketogenic fasting-mimicking diet, less than 30 g of proteins for each day of the immune-enhancing ketogenic fasting-mimicking diet, 15-30 grams of monounsaturated fats for each day of the immune-enhancing ketogenic fasting-mimicking diet, 2-10 g of polyunsaturated fats for each day of the immune-enhancing ketogenic fasting-mimicking diet, and less than 12 g of saturated fats for each day of the immune-enhancing ketogenic fasting-mimicking diet.

32. The method of claim 24, wherein the immune-enhancing ketogenic fasting-mimicking diet package includes rations formulated to provide 3.5 to 5 kcal per pound of the subject for day 1.

33. The method of claim 32, wherein the immune-enhancing ketogenic fasting-mimicking diet package includes rations formulated to provide less than 25 g of sugar, less than 23 g of proteins, 16-25 grams of monounsaturated fats, 4.8 to 8 g of polyunsaturated fats, and 1 to 10 g of saturated fats for day 1.

34. The method of claim 24, wherein the immune-enhancing ketogenic fasting-mimicking diet package includes rations formulated to provide a total calorie consumption of 2.4 to 4 kcal per pound of the subject for days 2, 3, 4, 5, or any additional days.

35. The method of claim 34, wherein the immune-enhancing ketogenic fasting-mimicking diet package includes rations formulated to provide less than 16 g of sugars, less than 15 g of protein, 8 to 12 g of monounsaturated fats, 2 to 4 g of polyunsaturated fats and 1 to 6 grams of saturated fats. for days 2, 3, 4, 5, or any additional days.

36. The method of claim 24, wherein the immune-enhancing ketogenic fasting-mimicking diet package includes soups, nutrition bars, crackers, an optional drink with a glucose substitute, olives, vitamin and mineral supplements, algal oil, and optional teas.

37. The method of claim 24, wherein the immune-enhancing ketogenic fasting-mimicking diet package includes nutrition bars and soups to be consumed for breakfast, lunch, and dinner.

38. The method of claim 37, wherein one or more FMD soups formulated to provide less than about 1 gram of saturated fat per serving, less than about 5 grams of trans fat per serving, less than about 10 grams of protein per serving, less than about 40 grams of carbohydrates per serving.

39. The method of claim 38, wherein each FMD soup is formulated from any combination of components selected from the group consisting of black beans, butternut squash, quinoa, tomatoes, mushrooms, white beans, brown beans, spinach, green tea extract, rice flour, onions, brown rice powder, carrots, inulin, leeks, olive oil, cabbage, potatoes, olives, peas, pumpkin, maltodextrin, and celery, chicory root fiber, sea salt, yeast, basil, parsley, garlic, rosemary extract, coriander, oregano, potato starch, potato flakes, zucchini squash, turmeric.

40. The method of claim 38, wherein each FMD soup is formulated from a first component selected from the group consisting of black beans, butternut squash, quinoa, tomatoes, mushrooms, white beans, brown beans, spinach, and combinations thereof and a second component selected from the group consisting of green tea extract, rice flour, onions, brown rice powder, carrots, inulin, leeks, olive oil, cabbage, potatoes, olives, peas, pumpkin, maltodextrin, and celery, chicory root fiber, sea salt, yeast, basil, parsley, garlic, rosemary extract, coriander, oregano, potato starch, potato flakes, zucchini squash, turmeric, and combinations thereof.

41. The method of claim 37, wherein the immune-enhancing ketogenic fasting-mimicking diet package includes a first FMD nutrition bar formulated to provide about bout 150 kcal to 350 kcal per serving, 10 to 35 gram of total fat per serving, less than about 8 grams of saturated fat per serving, less than about 5 grams of trans fat per serving, less than about 10 grams of protein per serving, and less than about 30 grams of carbohydrates per serving.

42. The method of claim 41, wherein the first FMD nutrition bar is formulated from a combination of macadamia nuts, honey, pecans, almonds, almond butter, coconut flour, sea salt, mixed tocopherols (vitamin E), citric acid, and/or ascorbic acid.

43. The method of claim 37, wherein the immune-enhancing ketogenic fasting-mimicking diet package includes a second FMD nutrition bar formulated to provide about bout 70 kcal to 120 kcal per serving, 3 to 10 grams of total fat per serving, less than about 5 grams of saturated fat per serving, less than about 5 grams of trans fat per serving, less than about 6 grams of protein per serving, and less than about 20 grams of carbohydrates per serving.

44. The method of claim 43, wherein the second FMD nutrition bar is formulated from a combination of inulin, almond butter, brown rice crispy, cocoa powder, almonds, chocolate chips, rolled oats, brown rice syrup, flaxseed oil, rice dextrin, grape juice, and salt.

45. The method of claim 24, wherein the immune-enhancing ketogenic fasting-mimicking diet package includes a drink composition that includes a glucose substitute.
